(19) United States
(12) Patent Application Publication
(10) Pub. No.: US 2002/0142293 A1
(43) Pub. Date: Oct. 3, 2002

(54) Enteroviral polynucleotides, methods of detecting enteroviruses and kits containing the polynucleotides

(75) Inventors: Radu Crainic, Jouy en Josas (FR); Valerie Caro, Saint Michel sur Orge (FR); Sophie Guillot, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: INSTITUT PASTEUR, Paris Cedex (FR)

(21) Appl. No.: 09/995,598

(22) Filed: Nov. 29, 2001

Related U.S. Application Data
(60) Provisional application No. 60/253,741, filed on Nov. 29, 2000.

(51) Int. Cl. C12Q 1/70; C12Q 1/68; C07H 21/04

(52) U.S. Cl. 435/5; 435/6; 536/23.72

(57) ABSTRACT

The present invention provides polynucleotides useful for detecting and/or typing Enteroviruses contained in a sample by amplification and/or hybridization and/or sequencing; and kits containing the polynucleotides.

### FIELD OF THE INVENTION

The present invention relates to polynucleotides and probes useful for detecting and/or serotyping enteroviruses.

### BACKGROUND OF THE INVENTION

Human enteroviruses (HEV) belong to the Picornaviridae family. They are major human pathogens and are associated with a broad spectrum of clinical features including acute respiratory illness, aseptic meningitis, meningoencephalitis, myocarditis, hand-foot-and-mouth disease, neonatal multi-organ failure and acute flaccid paralysis (Melnick, 1996). Outbreaks of disease associated with a single serotype of enterovirus are often reported (Gjoen et al., 1996, Kopecka et al., 1995) and represent a major public health problem. In 1998, an outbreak of enterovirus 71 infection caused hand-foot-and-mouth disease and herpangina in more than 100,000 individuals in Taiwan, with hundreds of deaths due to complications including encephalitis, aseptic meningitis, pulmonary oedema or hemorrhagic, acute flaccid paralysis and myocarditis (Ho et al., 1999). HEV are currently responsible for 80 to 92% of aseptic meningitis cases with an identified aetiologic agent (Rotbart, 1995).

The global eradication of poliomyelitis will be soon achieved, vaccination with live oral poliovirus vaccine will be stopped and the ecological niche taken by poliovirus will become vacant. Interest has increased in the circulation, detection, identification and evolutions of non-polio enteroviruses and the emergence of new epidemic strains. The traditional classification of enteroviruses is based on antigenic specificity, as determined by the serum neutralization assay, with all the disadvantages that this method implies.

Based on their antigenic properties, the original sixty-four HEV serotypes were initially grouped into Polioviruses (PV), Coxsackieviruses A (CA), Coxsackieviruses B (CB), Echoviruses (E), and the more recently identified Enterovirus (EV) 68 to 71. A new HEV classification based on molecular and biological data has recently been proposed as an alternative to the antigenic classification (Hyypia et al., 1997, Poyry et al., 1996). This classification groups enteroviruses into 5 species: 1)-PV, including poliovirus type 1, 2 and 3, 2)-HEV-A, including 11 Coxsackieviruses A and EV71; 3)-HEV-B, including all Coxsackieviruses B, all Echoviruses, EV69 and CA9; 4)-HEV-C including 11 other Coxsackieviruses A; and 5)-HEV-D including EV68 and EV70 (Pringle, 1999). The enteroviruses previously classified as E22 and E23, which were shown to group independently (Hyypia et al., 1992), now constitute a new genus Parechovirus (with two serotypes) in the Picornaviridae family (Miiyo & Pringle, 1998).

Enterovirus typing is required for several main reasons: i) to distinguish polio- from non-polio-enteroviruses in the context of poliomyelitis eradication, ii) to determine the relationship between enterovirus type and clinical syndrome, iii) to identify new enterovirus types or variants, iv) to analyze enteroviruses in neonates and immunodeficient patients, and v) to investigate enterovirus molecular epidemiology and phylogeny (reviewed in Muir et al., 1998). The agent responsible for HEV-induced diseases is currently identified by conventional virus isolation followed by neutralization with intersecting pools of type-specific antisera. Due to the large number of antigenically distinct serotypes, serotyping procedures are time-consuming, labor-intensive and costly. Moreover, the limited supply of reference type-specific sera, the limited number of serotypes, covered by the intersecting pools of sera currently available (LBM or RIVM), their “static” character (the inability to detect new antigenic variants or emerging serotypes) are also major drawbacks of neutralism typing (el-Sageyer et al., 1998). In addition, enteroviruses are frequently found to be “untypeable”.

In the light of recent developments in molecular biology, several assays based on the reverse transcription-polymerase chain reaction (RT-PCR) followed by nucleic acid hybridization or sequencing have been assessed as possible approaches for the identification of enteroviruses. Accordingly, genomic sequencing has made it possible to develop various molecular approaches for enterovirus identification and genealogical studies. The enterovirus genome is a single-stranded, positive RNA molecule, approximately 7,500 nucleotides long, including a 5′ and a 3′ non-coding region (NCR), and encompassing a single, long open reading frame. Sets of primers specific for highly conserved sequences in the 5′NCR or VP2 capsid protein-coding regions have been used to develop efficient methods for the rapid and sensitive detection of enteroviruses (reviewed by Romero, 1999). However, neither the 5′NCR nor the VP2 coding region (Oberste et al., 1998) can be used for enterovirus typing, due to a lack of correlation between the nucleotide sequence of these genomic regions and serotype. Accordingly, this use made it possible to detect HEV, but not to identify them beyond the genus level. VP1 is a capsid protein located mainly at the virion surface. It makes a large contribution to the constitution of neutralization antigenic sites. For this reason, the region of the genome encoding VP1 has been used to investigate the molecular evolution of poliovirus (Kew et al., 1995), to determine poliovirus genotypes (Balanant et al., 1991) and to develop poliovirus serotype-specific PCR primers (Kilpatrick et al., 1998). The nucleotide sequence of the entire VP1 coding region has recently been shown to correlate with serotype in enteroviruses (Oberste et al., 1999a) and to contain serotype-specific information in HEV (Oberste et al, 1999b) opening up possibilities for the development of molecular methods for the identification of traditional HEV serotypes and the newly designated HEV species (Pringle, 1999) and of molecular diagnostic reagents for serotype-specific enterovirus identification.

Here is presented a new approach for the molecular detection and/or serotyping of HEV and for epidemiological studies of HEV, involving the amplification of a genomic fragment encompassing the VP1-2C coding region with a single pair of enterovirus-specific primers. Restricted analysis of the 3′ third of the VP1-coding region showed a good correlation between nucleotide sequence and enterovirus serotype, for both classical reference strains and field isolates, over a 30-year period, and covering widely dispersed geographic regions. Especially, it is here demonstrated that a genomic segment of 364 nucleotides (relative to VP1 Mahoney, Kitamura et al, 1981) of the 3′ third of the VP1-coding region of HEV genomes contains enough serotype-specific information to be used for HEV serotype identification. This method may improve diagnosis of the diseases caused by Enterovirus infection. The amplification of this long genomic fragment may also be used as a rapid and efficient tool for studies of HEV molecular epidemiology and evolution.

To explore further the phylogenetic relationships between human enteroviruses and to develop new diagnostic approaches, the inventors designed a pair of generic primers for the study of a 1452 bp genomic fragment (relative to Mahoney poliovirus genome), including the 3′ end of VP1-, the 2A- and 2B-, and the 5′ moiety of 2C-coding region. Fifty-nine of the sixty-four prototype strains and forty-five field isolates of various origins, involving 21 serotypes; and six strains untypeable by standard immunological techniques, were successfully amplified with these primers. By determining the nucleotide sequence of the genomic fragment encoding the C-terminal third of the VP1 capsid protein the inventors developed a molecular typing method based on RT-PCR and sequencing. If field isolate sequences were compared to human enterovirus VP1 sequences available in databases, nucleotide identity score was, in each case, highest with the homotypic prototype (74.8 to 89.4%). Phylogenetic trees were generated from alignments of partial VP1 sequences by several phylogeny algorithms. In all cases, the new classification of enteroviruses into five identified species was confirmed and strains of the same serotype were always monophyletic. Analysis of the results confirmed that the 3′ third of the VP1 coding sequence contains serotype-specific information and can be used as the basis of an effective and rapid molecular typing method. Furthermore, the amplification of such a long genomic fragment, including non-structural regions, is straightforward and could be used to investigate genome variability and to identify recombination breakpoints or specific attributes of pathogenicity.

Accordingly, an RT-PCR amplicon including the 3′ third of the VP1 coding region was obtained for 59 of the 64 reference and all of the 45 field isolates, using an original single pair of universal HEV-specific primers, flanking the 1452 nucleotide VP1-2C coding region (relative to PV1-Mahoney strain). This is the first time that it has been possible to detect and to identify 92.2% of all known prototype HEV with a single pair of primers. In another study (Oberste et al., 1999a), a set of degenerate deoxyinosine-containing PCR primers designed to amplify the region encoding VP1 region was shown to be effective for only 65% of enterovirus prototype strains. The same authors (Oberste et al., 2000) had to design five different pairs of primers to amplify 54 different field isolates. In the present study, the 5 prototype strains not recognized by the pair of primers of the invention have characteristics unusual among HEV. Indeed, only 80% and 70% identity with the sense primers was observed for the CA5, CA19 and CA22 group and for the EV68 and EV70 groups, respectively. These strains are also known to have other characteristics different from those of the other HEV: the three CA viruses thrive only in suckling mice and the two EV constitute a separate species (HEV-D).

### BRIEF DESCRIPTION OF THE DRAWINGS

FIG. 1: Phylogenetic tree showing relationships between the 45 enterovirus field isolates, based on the C-terminal third of the VP1-coding region sequence alignment. The prototype enterovirus representative of each species are indicated in bold. The tree was constructed by the neighbor-joining method with the maximum-likelihood method of Kishino & Hasegawa (Kishino, H. and Hasegawa, M., 1989) with a transition/transversion ratio of 8.0 for the distance matrix. Numbers at nodes represent the percentage of 100 bootstrap pseudoreplicates. The human enterovirus species (Poliovirus, HEV-A, HEV-B, HEV-C and HEV-D) are indicated on the right side by vertical bars (distances between bars are arbitrary). Horizontal branch legends are proportional to the number of substitutions as indicated by the scale (the scale represents 0.1 nucleotide substitutions per site).

FIG. 2: Phylogenetic trees depicting genetic relationships between field human enteroviruses and prototype HEV-A (a), HEV-C + poliovirus (b), and HEV-B (c). Trees were based on sequence alignment of the C-terminal third of the VP1-coding region and constructed by neighbor-joining method with the maximum-likelihood method of Kishino & Hasegawa (Kishino, H. and Hasegawa, M., 1989) with a transistion/transversion ratio of 8.0 for the distance matrix. Numbers at node represent the percentage of 100 bootstrap pseudoreplicates. Branch lengths are proportional to the number of substitutions as indicated by the scale (the scale bar represents 0.1 nucleotide substitutions per site). Human enteroviruses sequenced in this work are shown in bold.

### SUMMARY OF THE INVENTION

An object of the present invention is to provide polynucleotides which are capable of amplifying and detecting an Enterovirus nucleic acid in an easy, rapid and specific manner with a high sensitivity. To be more specific, the present invention is to provide amplifying primers and also a capturing probe or a detecting probe for identifying the amplified product obtained by said primers whereby the problems encountered in the conventional clinical test methods for Enteroviruses have now been solved.

Accordingly, an object of the present invention is a purified polynucleotide selected from the group consisting of SEQ ID NO:1, SEQ ID NO:2, SEQ ID NO:3, SEQ ID NO:4, SEQ ID NO:5 and SEQ ID NO:6 or from the group consisting of SEQ ID NOS:7-51. These sequences include those sequences which stringently hybridizes to one or more of theses polynucleotides.

A further object of the present invention is a method of detecting an Enterovirus in a sample comprising contacting a polynucleotide region of a VP1-2C gene of said enterovirus with amplification primers; amplifying said polynucleotide region of the VP1-2C genes; and detecting the presence of an amplified polynucleotide, wherein the presence of the amplified product is indicative of the presence of the Enterovirus in the sample.

In such a method, the amplification comprises reverse transcription and is named RT-PCR.

Another object of the present invention is a method of detecting the presence of an Enterovirus in a sample, comprising: contacting an isolated and purified nucleic acid comprising (a) a polynucleotide sequence contained in the VP1-2C gene of said Enterovirus with the sample containing (b) an enterovirus; detecting the presence or absence of hybridization between (a) and (b), wherein the presence of hybridization between (a) and (b) is indicative of the presence of the enterovirus in the sample.

These polynucleotide sequences are selected from the group consisting of SEQ ID NO:7-51 or fragments thereof or sequences hybridizing with one or more of sequences SEQ ID NOS:7-51 or fragments thereof.

Another object of the present invention is a method of detecting the presence of an Enterovirus in a sample, comprising: contacting a polynucleotide region of VP1-2C genes of said enterovirus with amplification primers; amplifying said polynucleotide region of the VP1-2C genes; sequencing said amplified polynucleotide region; and comparing the sequence of said amplified polynucleotide region with known sequences of enterovirus of various serotypes, wherein an identity between score of at least 75% indicates that the two compared sequences are from enteroviruses of the same serotype.

In the present application, identity means that the nucleotides of the compared sequences are identical and identically placed in the sequences. An identity score (also called percent homology) of at least 75% between two sequences indicates that such sequences are from enteroviruses of the same serotype. The percent homology can be determined with the GCG sequence analysis software, Genetics Computer Group, Madison, Wis.

Another object of the present invention is to provide kits suitable for detecting and/or serotyping enterovirus. Such kits include one or more of polynucleotide sequences selected from the group consisting of SEQ ID NO:1, SEQ ID NO:2, SEQ ID NO:3, SEQ ID NO:4, SEQ ID NO:5, and SEQ ID NO:6; and SEQ ID NO:7-51.

Another object of the present invention is a purified polynucleotide of VP1-2C genes produced by the above-method with or without amplification primers and the purified polypeptides encoded by such a purified polynucleotide.

### DETAILED DESCRIPTION OF THE INVENTION

The present invention provides a method of detecting the presence of an enterovirus in a sample. The method comprises amplifying the viral RNA from the sample using a purified polnucleotide/purified nucleic acid capable of selectively hybridizing with the enterovirus nucleic acid said isolated polynucleotide being selected from the group of the nucleotide sequences set forth in the Sequence Listings as SEQ ID NOS:1-6 (the primers in Table 1: EUC2, EUC2a, EUC2b, EUG3a, EUG3b and EUG3c, respectively), or the sequence complementary thereto and detecting the presence of amplification, the presence of amplification indicating the presence of the Enterovirus in the sample. Detecting the presence of amplification can be conducted by any technique known in the art such as electrophoresis. In addition, the Enterovirus can be detected and/or serotyped by hybridization with one or more of SEQ ID NOS:7-51(enteroviruses 1-45, respectively in Tables 2 and 3) or a fragment of said sequence, such a fragment is preferably chosen in the 3′ third of VP1.

The terms “polynucleotide” and “nucleic acid” are used herein according to the meaning known in the art. For example, DNA and/or RNA molecules composed of various nucleotides and/or nucleotide analogs. When the molecule is composed of a number of nucleotides of less than 30, they can also be named oligonucleotides.

The term “fragments” as it relates to polynucleotides and nucleic acid molecules is understood to mean those fragments which are specific for enteroviruses when used for methods to identify the presence of an enterovirus and specific for an enterovirus serotype when used in a method for serotyping an enterovirus.

The term “molecular serotyping” as used herein is understood to mean serotyping by one of molecular techniques rather than one of usual immunological techniques.

A “polypeptide” as used herein is understood to mean a sequence of several amino acid residues linked by a peptide bond. Such amino acids are known in the art and encompass the unmodified and modified amino acids. In addition, the polypeptide may be modified by one or modifications known in the art such as glycosylation, phosphorylation, etc.

The enterovirus can also be detected and/or serotyped by sequencing of its VP1-2C genes and comparing the obtained sequences with known sequences of VP1-2C genes. Methods of sequencing nucleic acids are known in the art and are described in, for example, Sambrook et al., Molecular Cloning: A Laboratory Manual, Cold Spring Harbor Laboratory, New York (1989) and Current Protocols in Molecular Biology, Ausebel et al, eds., John Wiley and Sons, Inc., New York (2000).

Examples of Enteroviruses viruses capable of providing a target nucleic acid sequence for amplification and detection by the method of the present invention include polioviruses, group A coxsackieviruses, group B coxsackieviruses, echoviruses and other newly identified enteroviruses. Preferred Enterovirus DNA targets include the VP1 gene, and particularly the 3′ third of the VP1 gene.

The viruses can be isolated from any known species of animal which carry Enteroviruses. Preferably the animals are human. The viruses can be contained in any sample obtainable from the animal, for example, saliva, blood, urine and stool, muscle, liver, thymus, cerebrospinal fluid samples and any other product of animal origin. The samples can be subject to purifying protocols known in the art or used in the detection analysis directly. For example, column chromatography, density centrifugation, or ammonium sulfate precipitation. These and other methods are disclosed, for example, in Sambrook et al., Molecular Cloning: A Laboratory Manual, Cold Spring Harbor Laboratory, New York (1989). For simplicity and ease of assay, it is preferred that the sample be used directly without purification.

The nucleic acid specific for the Enteroviruses can be detected utilizing a nucleic acid amplification technique, such as polymerase chain reaction (PCR) or ligase chain reaction (LCR). Alternatively, the nucleic acid is detected utilizing direct hybridization or by utilizing a restriction fragment length polymorphism.

Hybridization can be carried out either using the RNA contained in the viruses or the viral genome may be reverse transcribed prior to amplification. PCR primers which hybridize only with nucleic acids specific for the Enterovirusare utilized. The presence of amplification indicates the presence of the virus. In another embodiment a restriction fragment of a nucleic acid sample can be sequenced, directly using techniques known in the art and described herein, and compared to the known unique sequence to detect the Enterovirus. The present invention also contemplates a method of detecting the presence of the Enterovirus by selective amplification by the methods described herein. Alternatively, the Enterovirus can be detected by directly hybridizing the unique sequence with a nucleic acid probe selective for the Enterovirus. Furthermore, the nucleotide sequence could be amplified prior to hybridization by the methods described above. Such hybridization protocols are known in the art and are disclosed, for example, in Sambrook et al., Molecular Cloning: A Laboratory Manual, Cold Spring Harbor Laboratory, New York (1989). Such a sequence is, for example, the sequence of an Enterovirus amplified by the primers having the sequences in SEQ ID NO:1-6.

As used herein stringent hybridization conditions are those conditions which allow hybridization between polynucleotides that are 75%, 80%, 85%, 90%, 95%, or 98% as determined using conventional homology programs, an example of which is UWGCG sequence analysis program available from the University of Wisconsin. (Devereaux et al., Nucl. Acids Res. 12: 387-397 (1984)). Such stringent hybridization conditions typically include washing the hybridization in 2×SSC and 0.5% SDS at 65° C. (Sambrook et al., Molecular Cloning: A Laboratory Manual, Cold Spring Harbor Laboratory, New York (1989)). Of course, one of skill in the art will recognize that conditions can be varied depending on the length of the polynucleotides to be hybridized and the GC content of the polynucleotides see, for example, Sambrook et al., Molecular Cloning: A Laboratory Manual, Cold Spring Harbor Laboratory, New York (1989).

Since Enterovirus have an RNA genome, it is preferred to first amplify the viral genetic material by reverse transcriptase PCR (RT-PCR) to generate a DNA template suitable for sequencing or hybridization. Primers useful for RT-PCR include primers SEQ ID NOS:1-6 which can amplify a region of the Enterovirus, and preferably, the VP1-2C region. Repeated cycles of denaturation, primer annealing and extension carried out with polymerase, e.g., a heat stable enzyme Taq polymerase, leads to exponential increases in the concentration of desired nucleic acid sequences. The nucleic acid can be denatured at high temperatures (e.g., 95° C.) and then reannealed in the presence of a large molar excess of oligonucleotide. The oligonucleotide, oriented with their 3′ ends pointing towards each other, hybridize to opposite strands of the target sequence and prime enzymatic extension along the nucleic acid template in the presence of the four deoxyribonucleotide triphosphates. The end product is then denatured again for another cycle. After this three-step cycle has been repeated several times, amplification of a nucleic acid segment by more than one million-fold can be achieved.

PCR may be followed by restriction endonuclease digestion with subsequent analysis of the resultant products. Nucleotide substitutions can result in the gain or loss of specific restriction endonuclease site. The gain or loss of a restriction endonuclease recognition site facilitates the detection of the organism using restriction fragment length polymorphism (RFLP) analysis or by detection of the presence or absence of a polymorphic restriction endonuclease site in a PCR product that spans the sequence of interest.

Primers for PCR, RT-PCR and LCR are usually about 20 bp in length and preferably are from 15 to 25 bp. Better amplification is obtained when both primers are the same length and with roughly the same nucleotide composition. Denaturation of strands usually takes place at 94° C. and extension from the primers is usually at 72° C. The annealing temperature varies according to the sequence under investigation. Examples of reaction times are: 20 mins denaturing; 35 cycles of 2 min, 1 min, 1 min for annealing, extension and denaturation; and finally, a 5 min extension step. Other conditions may be used and will depend on the target, primers, type of sample, etc. Amplification protocols are disclosed, for example, in Innis et al., PCR Protocols, a Guide to Methods and Applications, eds., Academic Press (1990)).

In this invention “primer” means a polynucleotide which is produced synthetically or biologically and includes a specific nucleotide sequence which permits hybridization to a section containing the target nucleotide sequence. Defined primers/polynucleotides may be produced by any of several well known methods, including automated solid-phase chemical synthesis using cyanoethylphosphoramidite precursors. Other well-known methods for construction of synthetic primers/oligonucleotides may, of course, be employed. 2 J. Sambrook, E. F. Fritsch and T. Maniatis, Molecular Cloning 11 (2d ed. 1989).

The primers used to amplify the sample nucleic acids may be coupled to a detectable moiety. A preferred example of such a detectable moiety is fluorescein, which is a standard label used in nucleic acid sequencing systems using laser light as a detection system. Other detectable labels can also be employed, however, including other fluorophores, radio labels, chemical couplers such as biotin which can be detected with streptavidin-linked enzymes, and epitope tags such as digoxigenin detected using antibodies. The primers may be modified whereby another nucleotide is added to or substituted for at least one nucleotide in the oligonucleotide. The term “add(ed)” means that nucleotide, oligo dGTP, oligo DATP, oligo dTTP, oligo dCTP, etc. having fluorescence substance, linker arm, biotin, etc. are bound to a 5′-terminal or a 3′-terminal of the oligonucleotide sequence. The term “substitute(d)” means that nucleotide having fluorescence substance, linker arm, biotin, etc. is introduced as a substitute for at least one nucleotide in the oligonucleotide. Introduction of known labels such as radioactive substances, enzymes, fluorescence substances, etc. after synthesis of oligonucleotide is also included therein.

Examples of suitable primers for reverse-transcription and amplification in the present method are shown in Table 1 (SEQ ID NOS:1-6).

For example, primers are from about 15 to 25 nucleotides in length, without internal homology or primer-primer homology. It is also desirable for the primers to form more stable duplexes with the target DNA at the primer's 5′-ends than at their 3′-ends, because this reduces false priming. Stability can be approximated by GC content, since GC base pairs are more stable than AT pairs, or by nearest neighbor thermodynamic parameters. Breslauer et al., “Predicting DNA duplex stability from base sequence”, Proc. Nat'l Acad. Sci. USA 83: 3746-3750 (1986).

Additional factors to the selection of primers for amplification are discussed in Rylchik, W., Selection of Primers for Polymerase Chain Reaction”, in Methods in Molecular Biology, Vol. 15: PCR Protocols: Current Methods and Applications, White, B. A. ed., Humana Press, Totowa, N.J., 1993. Primer pairs are selected by position, similarity of melting temperature, internal stability, absence of internal homology or homology to each other and the 3′-end will not form a stable hairpin loop back on itself.

To evaluate compatibility of primers for use in amplification, it is desirable to determine the predicted melting temperature for each primer. This can be accomplished in several ways. For example, the melting temperature (Tm) can be calculated using either of the following equations:

Tm(°C.)=81.5+16.6×log[Na]+0.41×(% GC)−675/length

where [Na] is the concentration of sodium ions, and the % GC is in number percent,

or

Tm(°C.)=2×(A+T)+4(G+C)

where A, T, G, and C represent the number of adenosine, thymidine, guanosine and cytosine residues in the primer. Preferably, primers for coamplification should be selected to have predicted melting temperatures differing by less than 4° C.

The present invention employs one or more polymerizing enzymes capable of polymerizing deoxyribonucleoside triphosphates (and modified deoxyribonucleoside triphosphates) into a complementary strand of DNA using an amplification primer and a target nucleic acid sequence as a template. Examples of such enzymes are Avian Myeloblastosis Virus Reverse Transcriptase (AMV-RT), Moloney Murine Leukemia Virus Reverse Transcriptase (MMLV-RT), Superscript II (Life Technologies, Inc., Rockville, Md.), and DNA polymerases such as E. coli DNA polymerase, E. coli DNA polymerase (Klenow fragment), ^{exo-}E. coli DNA polymerase (Klenow fragment), Bst DNA polymerase, Taq DNA polymerase, Tfl DNA polymerase, and Tth DNA polymerase.

The amplification reaction may be carried out for a limited number of amplification cycles. It will be understood, that the more cycles of amplification are carried out, the more of the desired product will be made and thus the easier its detection will be. It should also be recognized, however, that during the initial cycles (generally the first 20-25 cycles), the amount of DNA of the desired sequence doubles in each cycle, while thereafter the yield of desired product per cycle drops off. For maximum effectiveness in the method of the present invention, the amplification of the target nucleic acid should be carried out only for a number of cycles during which doubling of DNA is still being achieved.

The present invention further features a kit that incorporates the components of the invention and makes possible convenient performance of the invention. Such a kit may also include other materials that would make the invention a part of other procedures, and may also be adaptable for multi-well technology. The kit is useful for amplification, detection, typing, or combination thereof for the Enteroviruses presumed to be contained in a sample.

Preferred components of the kit(s) include any one of the inventive polynucleotide sequences, either the primers used for amplification possibly comprising reverse transcription, e.g., SEQ ID NOS:1-6 or the polynucleotide containing the sequence amplified, e.g, SEQ ID NO:7-51 or fragments thereof, such fragments being specific of the serotype when the kit is used for serotyping of enteroviruses and specific for enteroviruses when used for methods to identify the presence of an enterovirus in a sample. Other suitable kit components include, enzymes used in amplification or hybridization, buffers for diluting the sample to be tested, and reagents for detecting the amplified products and/or hybridized samples. The polynucleotides contained in the kit can be modified with detectable moieties prior to inclusion into the kit or include reagents suitable for modifying the polynucleotides with detectable moieties as is known in the art.

To avoid missing the detection of an enterovirus by our RT-PCR assay, an alternative mean of detection should be used. One possibility is to include in the diagnosis procedure the classical enterovirus-specific PCR in the 5′NCR. If detected in this way, an enterovirus which was not amplified with the pair of primers of the invention could be further genotyped with an HEV-D or CA5, 19 and 22-specific primers.

### EXAMPLES

Having generally described this invention, a further understanding can be obtained by reference to certain specific examples which are provided herein for purposes of illustration only, and are not intended to be limiting unless otherwise specified.

Methods

Prototype Viruses

The Echoviruses, Coxsackieviruses B, Coxsackieviruses A and enterovirus types 68 to 71 studied in this work were the ‘prototype’ strains of each serotype (Melnick, 1996) and were kindly supplied by the National Institute of Public Health and the Environment (RIVM), Bilthoven, The Netherlands. The serotype of the reference strains was checked by seroneutralization using the Lim Benyesh-Meinick (LBM) panel of intersecting antisera pools (Melnick et al., 1973).

Field Isolates

Forty-five enterovirus isolates were selected, representing 21 serotypes throughout the Enterovirus genus. The panel included 35 virus isolates from Europe (France, Greece, the Netherlands and Romania) and 10 isolates from Africa (Burkina Faso, Madagascar) (Table 2). They were isolated from original clinical specimens: stool (n=31), cerebrospinal fluid (n=4), blood (n=1), cortex (n=1), spinal cord (n=2), nasopharyngeal secretion (n=2), vesicle (n=2) and throat (n=2). Clinical symptoms and the specimens studied in this work are described in Table 2. The serotypes of Romanian isolates were determined by neutralization with the LBM and home-made antisera pools and those of the Dutch, French, Greek, Madagascar isolates were determined by neutralization with the RIVM pools (Kapsenberg, 1988). The 6 Madagascan enterovirus isolates, which were not neutralized by any of the PDVM pools, were classified as “untypeable”.

RT-PCR

RNA was extracted from 100 μl of infected cell culture supernatant using the Total Quick RNA Talent Idt (Euromedex, France) and eluted in 70 μl of DEPC-treated water. RNA (2 μl) was used for cDNA synthesis with 10 pmol each of the antisense primers, EUC2a and EUC2b (see Table 1 for the primers, SEQ ID NOS:1-6). The reaction (20 μl) contained 50 mM Tris-HCl pH 8.3, 75 mM KCl, 3 mM MgCl₂, 10 mM dNTPs, 2 mM DTT, 20 U ribonuclease inhibitor (RNasin, Promega) and 200 U Superscript RNase H-reverse transcriptase (Gibco BRL, Life Technologies). The reaction mixture was incubated for 30 min at 42° C. and was then heated for 5 min at 95° C. to inactivate the enzyme. The cDNA product (2 μl) was added to a PCR mixture containing 67 mM Tris-HCl pH 8.8, 16 mM (NH₄)₂SO₄, 0.01% Tween-20, 2 mM MgCl₂ 10 mM dNTPs, 1.25 U of Taq DNA polymerase (EurobioTaq, Eurobio) and 10 pmol of primers (EUC2, EUG3a, EUG3b and EUG3c). Amplification involved 29 cycles of denaturation at 95° C. for 20 s, annealing at 45° C. for 1 min and elongation at 72° C. for 1 min, followed by a final cycle of denaturation at 95° C. for 20 s, annealing at 45° C. for 1 min and elongation at 72° C. for 10 min. Amplification products (5 μl) were run on 1.5% agarose gels. The gels were stained with ethidium bromide and the DNA was viewed under UV light. RT-PCR was used to amplify a 435 bp fragment in the 5′ non-coding region, from 2 μl of purified RNA, as previously described (Balanant et at., 1991), using primers UC52-UG53 (Gufflot et al., 1994).

Nucleotide Sequence Determination and Sequence Analysis

PCR products were purified by the low-melting-point agarose gel method (Sambrook et al., 1999) and sequenced on an automated DNA sequencer using the BigDye Terminator Cycle Sequencing Ready Reaction kit (Perldn Elmer Biosystems), with the EUG3a, EUG3b and EUG3c primers. Sequences were compared with the GenBank database sequences using the program FASTA version 3.3 (Pearson & Lipman, 1988).

Pairwise nucleotide and amino acid sequence identities were calculated by aligning each field isolate sequence with each available prototype enterovirus sequence using the multiple alignment program CLUSTAL W (Thompson et al., 1994). To analyze phylogenetic relationships, the partial VP1 sequences of isolates were compared with those from other human enteroviruses using CLUSTAL W. Some reference enterovirus nucleotide sequences from the GenBank database were used (Oberste et al., 1999b). Alignments were coffected manually to maximize sequence identity, to account for codon boundaries and to ensure the alignment of conserved amino acid motifs. Phylogenetic trees were generated by inputting the aligned sequences into PHYLIP (Phylogeny Inference Package) version 3.5 (Felsenstein, 1993) and PUZZLE version 4.0 (Strimmer & von Haeseler, 1996). Phylogenetic trees were constructed using the neighbor-joining algorithm of Saitou & Nei (1987), as implemented in the program NEIGHBOR, and using the maximum parsimony method, as implemented in DNAPARS. For neighbor-joining analysis, a distance matrix was calculated using the Kishino and Hasegawa method (Kishino & Hasegawa, 1989) with a transition/transversion ratio (k) of 8.0, using DNADIST (PHYLIP). The k parameter is an empirical ratio calculated by PUZZLE from the data set. To investigate the robustness of the phylogenies constructed with NEIGHBOR and DNAPARS, bootstrap analysis was carried out on 100 pseudo-replicate data sets with SEQBOOT. Phylogenic trees were reconstructed by the maximum likelihood method with PUZZLE, which uses QUARTET PUZZLING as the tree search algorithm. Distances were calculated with the model of nucleotide substitutions of Kishino and Hasegawa (Kishino & Hasegawa, 1989) and the transition/inversion parameter was estimated directly from the data set. The reliability of tree topology was estimated by use of 1,000 puzzling steps. The trees were drawn using program TREEVIEW (Page, 1996).

Nucleotide Sequence Accession Numbers

The nucleotide sequence data reported in the paper amplification have been submitted to the EMBL sequence database under the accession numbers AJ279 151 to AJ279195 (SEQ ID NOS:7-51 Table 2).

### Results

Selection of Generic Primers and Evaluation for Amplification

The first goal was to develop a RT-PCR assay capable of detecting all known serotypes of human enteroviruses, facilitating the identification of enteroviruses and phylogenetic study by means of amplicon sequencing. The inventors therefore designed a pair of generic degenerate PCR primers, binding to the sequences on either side of the sequences encoding, VP1 and 2C (Table 1, SEQ ID NOS:1-6). None of the antisense primers contained a mixed-base and none of the sense primers had more than one degenerate position. The amplicon thus obtained was 1452 bp long (relative to the PV I-Mahoney sequence) and included the 3′ end of the VP1 coding sequence, the entire coding sequence of 2A and 2B, and the 5′ moiety of the 2C coding region of the enterovirus genome.

To determine specificity, an equimolar mixture of primers was first tested in an RT-PCR assay with the RNA extracted from each of the prototype human enteroviruses. Amplicons were obtained from 59 of the 64 prototype human enteroviruses (92.2%). The viruses for which the amplification reaction was unsuccessful were CA5, CA19 and CA22, which thrive only in suckling mice, EV68 and EV70, members of the distant HEV-D species, and E22 and E23, which are now classified as a new genus (Parechovirus) of the Picornaviridae family (Hyypia et al., 1992). The failure of amplification was not due to a low concentration of viral RNA in the reaction, as shown by the successful amplification of an HEV-specific genomic fragment from the 5′ NCR, as for all other HEV strains tested (not shown).

Amplicons were also successfully obtained from all of the 45 clinical enterovirus isolates tested (Table 3), irrespective of their date of isolation (1970 to 1998), serotype (21 different serotypes), or the geographical region in which they were collected.

Nucleotide Sequence and Serotypes

We sequenced 600-700 nucleotides including the 3′ third of the VP1 gene and the beginning of the 2A gene for each isolate using sense EUG3a, EUG3b and EUG3c primers in combination or separately. All new sequence data presented in this work have been published in the EMBL database (Table 2). The partial VP1 sequence of each of the 45 field isolates (a stretch of 365 nucleotides from 3021 to 3385, numbering according to PV1-Mahoney) was compared with all enterovirus sequences published in GenBank. The gbvrl library was used for nucleic acid analysis and the gpvrl library for protein analysis gbvrl and gpvrl libraries are options of Genbank. A hundred percent correlation was obtained between the serotype determined by the 3′ third of the VP1 coding region sequence and the serotype determined by the conventional neutralization assay for all the 45 field isolates of known serotype tested (Table 3).

For each field isolate tested, identity was highest with the homotypic prototype strain, for both nucleotide and amino acid sequences, with identities of 74.8 to 89.4% for nucleotide sequences and from 89.8 to 98.2% for amino acid sequences (Table 3). In all cases in which a sequence from a more recent homotypic isolate was present in the database, a better score was obtained with this strain than with the homotypic reference strain (not shown). However, in one case (CB5-RO-14/5/70), the VP1 sequence of the isolate was less similar to the homotypic CB5 prototype (78.5%) than to the prototype of swine vesicular disease virus (SVDV), a pig enterovirus (89.2%), with which the highest identity score was obtained. The deduced amino acid sequence of CB5-RO-14/5/70 was 95.5% similar to those of both SVDV and CB5. This is not surprising as it has been shown that SVDV (Swine Venicular Disease virus) probably arose in pigs from a single transfer of a human CB5, and may therefore be considered to be a subspecies of CB5 (Zhang et al., 1999).

In all cases, the second-highest identity scores with respect to another serotype were 67.9 to 78.5% for nucleotide sequences and 70.4 to 95.5% for amino acid sequences. In each case, the prototype strain giving the second-highest score belonged to the same species as the strain giving the highest score. The range of the delta scores, representing the difference in percentage nucleotide sequence identity between the highest and second highest scores was from 1.4 to 19.4% (Table 3). This difference, demarcating the boundary between serologically homotypic and the closest heterotypic strains, was on average 10.0% but was very low for seven clinical isolates. Isolate E1-RO-122/1/74 had a nucleotide sequence 80.5% similar to that of E1 prototype strain and 77% similar to that of E8 prototype strain. This difference of only 3.5% is consistent with the previously demonstrated antigenic relationship between E1 and E8 (Harris et al., 1973) and the reclassification of E8 as a variant of E1. The other six isolates with a small difference in percentage nucleotide identity between the highest and second-highest scores (1.4 to 2.9%) are, discussed below.

Analysis of the “Untypeable” Field Enteroviruses

Strains MG-354/94, MG-356/94, MG-404/94, MG-448/94, MG-423/94 and MG498/94 were all isolated from the stools of healthy Malagasy children (Table 2). They were identified as enteroviruses by their cytopathic effect on an enterovirus-susceptible cell line and by the enterovirus-specific amplification (RT-PCR) of the 5′ non-coding genomic region. They were not neutralized by intersecting RIVM pools. Their genome was successfully amplified by our pair of enterovirus-specific VP1-2C primers and sequenced. The highest and second-highest identity scores obtained by comparing the partial VP1 sequences of these strains with those of HEV strains in the GenBank are reported in Table 3. Isolates MG-354/94, MG-356/94, MG-404/94 and MG-498/94 were 77.4 to 80.2% identical to CA13 (Flores) and 75.4 to 78.0% identical to CA18 (G-13) prototype strains. The high level of sequence identity between these two reference strains (76.0%), consistent with their antigenic relatedness (Committee on Enteroviruses, 1962), may account for the small difference between the two scores (1.4 to 2.2%). By neutralization with several monospecific CA13 antisera (kindly supplied by RIVM), all the above isolates were identified as serotype CA13 (not shown). A similar problem of a small difference between the highest and second-highest scores was encountered for strains MG-448/94 and MG-423/94. Indeed, MG-448/94 was 74.8% identical to CA20 (IH-35) and 71.9% identical to CA17 (G-12) reference strains, with the CA20 and CA17 prototypes displaying only 70.0% sequence identity. Similarly, strain MG-423/94 was 75.0% identical to CA20 and 72.3% identical to CA13, with the CA20 and CA13 prototypes only 71.0% identical to one another. No antigenic cross-reactivity of CA20 with CA17 or CA13 has been reported. Both isolates were identified as CA20 isolates by neutralization with monospecific sera. However, they were strongly neutralized by both anti-CA20 (IH-35) and anti-CA20a (Tulano) antibodies, and less strongly by anti-CA20b (Cecil) antibodies (not shown), reflecting the antigenic relationship of these three variants of CA20 (personal communication from Albert Ras). Thus, in this study, isolates MG-448/94 and MG-423/94 had the lowest percentage nucleotide sequence identity with the closest reference prototype strain (74.8 and 75% respectively). These field strains have probably, accumulated several mutations, causing them to drift away from their homologous prototype, which was isolated in 1955.

Phylogenetic Relationships of Field Isolates

To determine the relationships between field and prototype HEV, the sequence of the 3′ third of the VP1-coding region of each field isolate was compared with those of all prototype strains by pairwise alignments, with the program CLUSTAL W. In each case, the homologous serotype pairwise comparison scores were higher than 75% for nucleotide identity and higher than 85% for amino acid identity. Furthermore, there is no overlap between the homologous serotype pairwise comparison scores of both nucleotide and amino acid sequences and the heterologous serotype pairwise comparison scores (data not shown). This confirmed the accuracy of the molecular serotyping: the serotype of every isolate could be determined if the 3′ third of VP1 sequence displayed a minimum of 75% nucleotide identity (85% amino acid identity) with a prototype strain in the database.

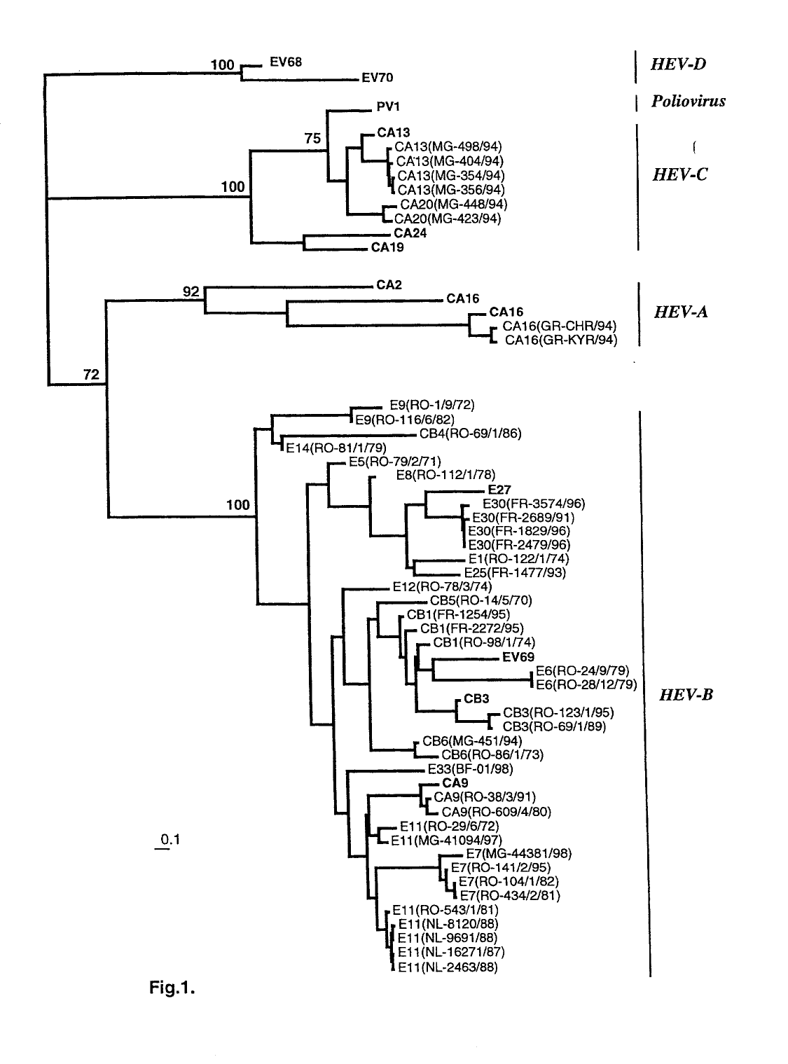

To explore further the evolutionary relationships between field and prototype viruses, a general tree was generated with the 45 isolates and representatives of each of the five identified HEV species (PV:PV1; HEV-A: CA2, CA12 and CA16; HEV-B: E27, CB3, CA9 and EV69; HEV-C: CA13, CA19 and CA24; HEV-D: EV68 and EV70). The same tree topology was produced, regardless of the algorithm used. The isolates clearly segregated into five distinct major groups (FIG. 1), consistent with previously published human enterovirus phylogenies (Huttunen et al., 1996, Oberste et al., 1998, Poyry et al., 1996, Pulli et al., 1995) and the new classification. The various groups were strongly supported by bootstrap values of 100% for HEV-B, C and D and 92% for HEV-A species, regardless of the algorithm used.

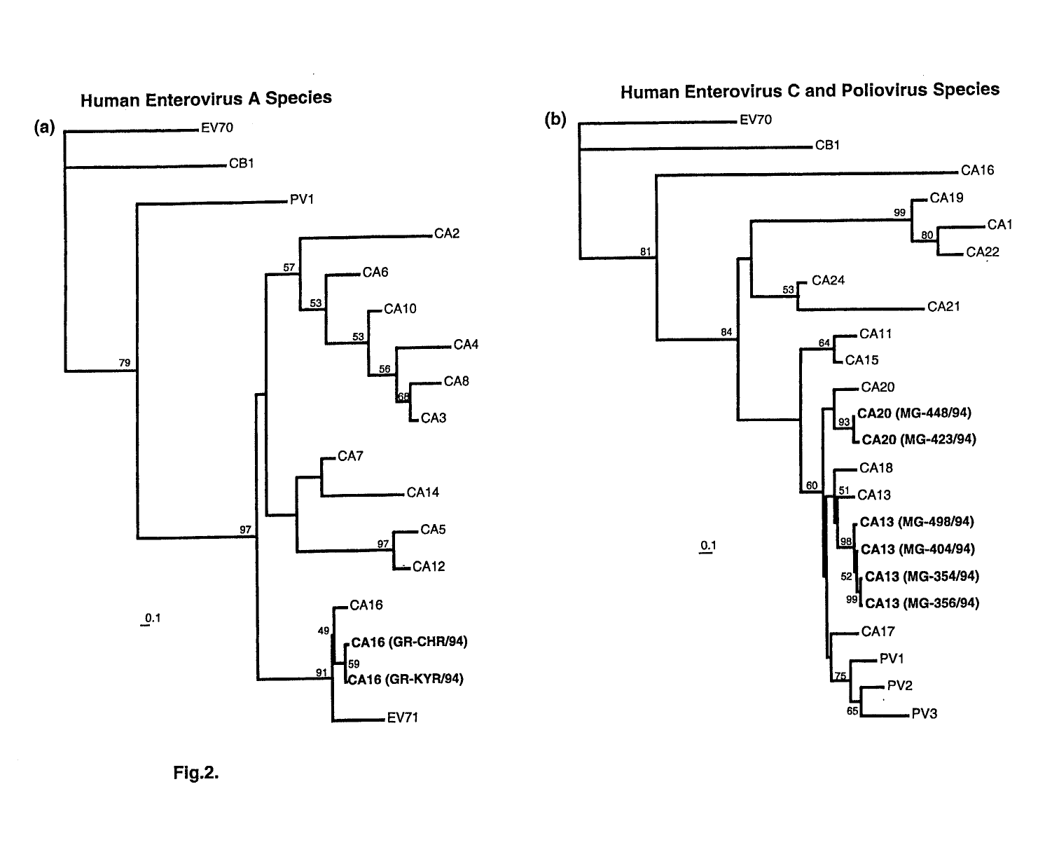

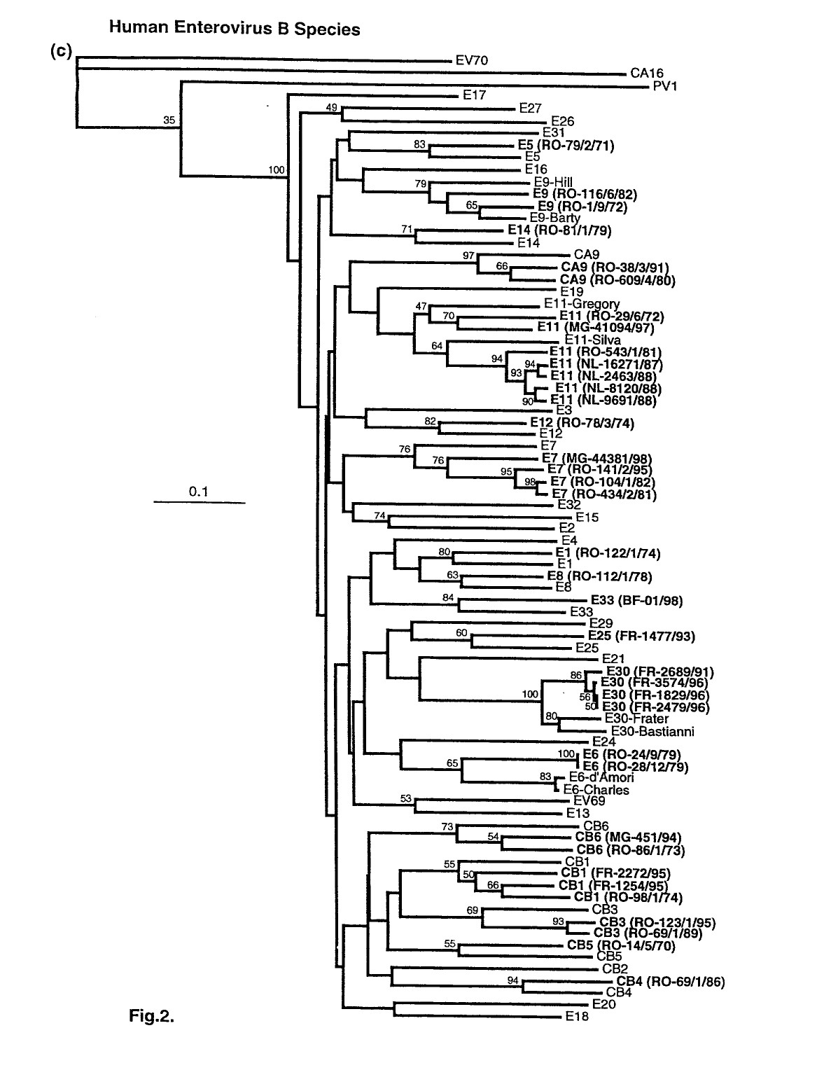

To study more precisely the phylogenetic relationships between field isolates, we constructed phylogenetic trees within each defined species for the clinical isolates and the corresponding prototype HEV. In each tree, one sequence from each of the other species was included as an outgroup. The HEV-D species was not analyzed because no appropriate isolate was available. Within each species, the same general tree topology was obtained irrespective of the method used. However, some variation in the branching order of some subgroups, in bootstrap values for certain nodes and in branch lengths was observed. Whatever the species, all homotypic strains (field and prototype) were monophyletic, as supported by parametric bootstrap analysis. Three subgroups, slightly different from the published subgroups (Oberste et al., 1999b) were observed for HEV-A species (FIG. 2a): (1) CA2, CA6, CA10, CA4, CA8 and CA3; (2) CA7, CA14, CA5 and CA12 and (3) EV71 and the two CA16 isolates. The CA16 isolates were closely related to each other and to their homologous prototype strain. The clustering of CA16 with the prototype EV71 strain (bootstrap value of 91%), is consistent with a prior observation that CA16 has an antigen in common with EV71 (Hagiwara et al., 1978). The HEV-C species was divided into the same 4 subgroups as already published (Oberste et al., 1999b): (1) CA1, CA19 and CA22; (2) CA21 and CA24; (3) CA11 and CA15 and (4) PV1, PV2, PV3, CA17, CA13 prototype isolates, CA18, CA20 prototype and isolates (FIG. 2b). HEV-B species made up the largest group, with 37 isolates of the 45 analyzed belonging to this phylogenetic group. By analyzing 37 field strains and 44 prototype strains, including three outgroup strains (EV70, CA16 and PV1) (FIG. 2c), we found that the field isolates formed monophyletic clades with their prototype, well supported by bootstrap analysis. All the CBVs clustered together, with each isolate close to its prototype. As previously reported, CB2 and CB4 were more related to each other than to the other CB serotypes, whereas the CB1, CB3 and CB5 strains constituted a single subgroup and CB6 formed a branch of its own (Lindberg & Polacek, 2000). In general, the clinical isolates of a given serotype were more closely related to each other than to their homologous prototypes, which were often displayed on a different branch.

The two E9 prototypes were not clustered together in a single subgroup and the E9RO-1/9/72 isolate was more closely related to the E9/Barty/57 prototype than to the other isolate, E9-RO-116/6/82. The E9-RO-1/9/72 isolate and E9/Barty/57 were very similar. E9-RO-1/9/72 was isolated from the spinal cord of a child with encephalitis and E9/Barty/57 was isolated from the cerebrospinal fluid of a child with aseptic meningitis. The two E9 field strains had an additional 10 amino-acid fragment including an RGD motif in the C-terminal part of the VP1 structural protein (data not shown), which differentiated the pathogenic Barty strain from the non-pathogenic Hill strain (Zimmerman et al., 1996), and has been described as a probable major determinant of virulence (Nelsen-Salz et al., 1999, Zimmermann et al., 1997).

The neutralization test, the traditional standard procedure for enterovirus identification, is generally reliable but may fail to identify isolates due to mixtures of enteroviruses, the aggregation of virus particles, antigenic drift, or simply because it is impossible to identify all circulating HEV serotypes with the intersecting pools of antisera in current use. With the present system, it was possible to obtain amplicons from all of the 45 field strains, randomly chosen from HEV isolates representing 21 different serotypes, from various geographic regions of the world, spanning a 30-year period. By comparing the 3′ end of VP1 sequence of each isolate with those of all prototype enteroviruses, it was possible to confirm or to identify unambiguously the serotype of all these isolates. Six of the 45 isolates were untypeable with intersecting sera, but were correctly “serotyped” with the present molecular method, the results being confirmed by neutralization with monospecific antisera. It is also possible to determine the serotypes of over one hundred other strains, from 26 different serotypes (not shown).

The results reported here are consistent with previous findings (Oberste et al., 1999a), showing a good correlation between molecular and antigenic serotyping for HEV. The sequencing of VP1-2C coding region and especially of the 3′ third of the VP1 coding region, or its hybridization with a specific probe is a useful tool for the rapid identification of enteroviruses, for the diagnosis of enterovirus infections, for determining the extent of genotype divergence among isolates of a given serotype and for phylogenetic studies of enteroviruses. Thus, the molecular strategy improves the identification and characterization of enterovirus isolates and constitutes a rational basis for replacing serotyping by easy rapid genotyping.

The key advantage of the present molecular strategy for the identification of HEV, based on the use -of VP1-2C primers, is that it requires only a single pair of optimized polynucleotides for the serotyping and genotyping of almost all HEV strains, opening up new possibilities for diagnosis purposes, for studying epidemiological or pathological features and for searching for new serotypes of HEV.

Obviously, numerous modifications and variations on the present invention are possible in light of the above teachings. It is therefore to be understood that within the scope of the appended claims, the invention may be practiced otherwise than as specifically described herein.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

### REFERENCES

Badorff, C., Lee, G. H., Lamphear, B. J., Martone, M. E., Campbell, K. P., Rhoads, R. E. & Knowlton, K. U. (1999). Enteroviral protease 2A cleaves dystrophin: evidence of cytoskeletal disruption in an acquired cardiomyopathy [see comments]. Nature Medicine 5, 320-6.

Balanant, J., Guillot, S., Candrea, A., Delpeyroux, F. & Crainic, R. (1991). The natural genomic variability of poliovirus analyzed by a restriction fragment length polymorphism assay. Virology 184, 645-654.

Committee on Enteroviruses (1962). Classification of Human Enteroviruses. Virology 16, 501-504.

el-Sageyer, M. M., Szendroi, A., Hutter, E., Uj, M., Szucs, G., Mezey, I., Toth, I., Katai, A., Kapiller, Z., Pall, G., Petras, G., Szalay, E., Mihaly, I., Gourova, S. & Berencsi, G. (1998). Characterisation of an echovirus type 11′ (prime) epidemic strain causing haemorrhagic syndrome in newborn babies in Hungary. Acta Virologica 42, 157-66.

Felsenstein, J. (1993). PHYLIP: phylogeny inference package, version 3.5c (computer program). Distributed by the author. Department of Genetics, University of Washington, Seattle.

Gjoen, K., Bruu, A. L. & Orstavik, 1. (1996). Intratypic genome variability of echovirus type 30 in part of the VP4NP2 coding region. Archives of Virology. 141, 901-8.

Guillot, S., Otelea, D., Delpeyroux, F. & Crainic, R. (1994). Point mutations involved in the attenuation/neurovirulence alternation in type 1 and 2 oral polio vaccine strains detected by site-specific polymerase chain reaction. Vaccine 12, 503-507.

Hagiwara, A., Tagaya, I. & Yoneyama, T. (1978). Common antigen between coxsackievirus A 16 and enterovirus 71. Microbiol Immunol 22, 81-8.

Harris, L. F., Haynes, R. E., Cramblett, H. G., Conant, R. M. & Jenkins, G. R. (1973). Antigenic analysis of echoviruses 1 and 8. J Infect Dis 127, 63-8.

Ho, M., Chen, E. R., Hsu, K. H., Twu, S. J., Chen, K. T., Tsai, S. F., Wang, J. R. & Shih, S. R. (1999). An epidemic of enterovirus 71 infection in Taiwan. Taiwan Enterovirus Epidemic Working Group [see comments]. New England Journal of Medicine 341, 929-35.

Hughes, P. J. & Stanway, G. (2000). The 2A proteins of three diverse picornaviruses are related to each other and to the H-rev107 family of proteins involved in the control of cell proliferation. Journal of General Virology 81, 201-7.

Huttunen, P., Santti, J., Pulli, T. & Hyypia, T. (1996). The major echovirus group is genetically coherent and related to coxsackie B viruses. J Gen Virol 77, 715-25.

Hyypia, T., Horsnell, C., Maaronen, M., Khan, M., Kalkkinen, N., Auvinen, P., Kinnunen, L. & Stanway, G. (1992). A distinct picornavirus group identified by sequence analysis. Proceedings of the National Academy of Sciences of the United States of America 89, 8847-51.

Hyypia, T., Hovi, T., Knowles, N. J. & Stanway, G. (1997). Classification of enteroviruses based on molecular and biological properties. Journal of General Virology 78, 1-11.

Kapsenberg, J. G. (1988). Picornaviridae: The Enteroviruses (Polioviruses, Coxsackieviruses, Echoviruses). In Liboratory Diagnosis of Infectious Diseases. Principles and Practice. Volume II. Viral, Rickettsial and Chlamydial Diseases., pp. 692-722. Edited by P. H. Edwin H. Lennette, and Frederick A. Murphy. New York: Springer-Verlag.

Kew, O. M., Mulders, M. N., Lipskaya, G. Y., da Silva, E. E. & Pallansch, M. A. (1995). Molecular epidemiology of polioviruses. Semin. ViroL 6, 401-414.

Kilpatrick, D. R., Nottay, B., Yang, C. F., Yang, S. J., Dasilva, E., Penaranda, S., Pallansch, M. & Kew, O. (1998). Serotype-specific identification of polioviruses by PCR using primers containing mixed-base or deoxyinosine residues at positions of codon degeneracy. Journal of Clinical Microbiology 36, 352-357.

Kishino, H. & Hasegawa, M. (1989). Evaluation of the maximum likelihood estimate of the evolutionary tree topologies from DNA sequence data, and the branching order in hominoidea. Journal of Molecular Evolution 29, 170-9.

Kopecka, H., Brown, B. & Pallansch, M. (1995). Genotypic variation in coxsackievirus B5 isolates from three different outbreaks in the United States. Virus Res 38, 125-36.

Lindberg, A. M. & Polacek, C. (2000). Molecular analysis of the prototype coxsackievirus B5 genome. Arch Virol 145, 205-21.

Mateu, M. G. (1995). Antibody recognition of picornaviruses and escape from neutralization: a structural view. Virus Research 38, 1-24.

Mayo, M. A. & Pringle, C. R. (1998). Virus taxonomy—1997. Journal of General Virology 79, 649-57.

Melnick, J. L. (1996). Enteroviruses: Polioviruses, Coxsackieviruses, Echoviruses, and Newer Enteroviruses. In Fields Virology, third edition, pp. 655-712. Edited by D. M. K. B. N. Fields, P. M. Howley, R. M. Channock, J. L. Melnick, T. P. Monath, B. Roizman, and S. E. Straus. Philadelphia: Lippincott-Raven Publishers.

Melnick, J. L., Rennick, V., Hampil, B., Schmidt, N. J. & Ho, H. H. (1973). Lyophilized combination pools of enterovirus equine antisera: preparation and test procedures for the identification of field strains of 42 enteroviruses. Bulletin of the World Health Organization 48, 263-8.

Muir, P., Kammerer, U., Korn, K., Mulders, M. N., Poyry, T., Weissbrich, B., Kandolf, R., Cleator, G. M. & van Loon, A. M. (1998). Molecular typing of enteroviruses: current status and future requirements. The European Union Concerted Action on Virus Meningitis and Encephalitis. Clinical Microbiology Reviews 11, 202-27.

Nelsen-Salz, B., Schilling, O., Klein, M., Hadaschik, D., Eggers, H. J. & Zimmermann, H. (1999). Determinants of pathogenicity of echovirus 9 in men: significance of a functional RGD-motif. Zentralbl Bakteriol 289, 347-54.

Oberste, M. S., Maher, K., Flemister, M. R., Marchetti, G., Kilpatrick, D. R. & Pallansch, M. A. (2000). Comparison of classic and molecular approaches for the identification of untypeable enteroviruses. J Clin Microbiol 38, 1170-4.

Oberste, M. S., Maher, K., Kilpatrick, D. R., Flemister, M. R., Brown, B. A. & Pallansch, M. A. (1999a). Typing of human enteroviruses by partial sequencing of VP1. Journal of Clinical Microbiology 37, 1288-93.

Oberste, M. S., Maher, K., Kilpatrick, D. R. & Pallansch, M. A. (1999b). Molecular evolution of the human enteroviruses: correlation of serotype with VP1 sequence and application to picomavirus classification. Journal of Virology 73, 1941-8.

Oberste, M. S., Maher, K. & Pallansch, M. A. (1998). Molecular phylogeny of all human enterovirus serotypes based on comparison of sequences at the 5′ end of the region encoding VP2. Virus Research 58, 3543.

Page, R. D. (1996). TreeView: an application to display phylogenetic trees on personal computers. Computer Applications in the Biosciences 12, 357-8.

Pearson, W. R. & Lipman, D. J. (1988). Improved tools for biological sequence comparison. Proceedings of the National Academy of Sciences of the United States of America 85, 2444-8.

Poyry, T., Kinnunen, L., Hyypia, T., Brown, B., Horsnell, C., Hovi, T. & Stanway, G. (1996). Genetic and phylogenetic clustering of enteroviruses. Journal of General Virology 77, 1699-717.

Pringle, C. R. (1999). Virus taxonomy at the XIth International Congress of Virology, Sydney, Australia, 1999. Archives of Virology 144, 2065-70.

Pulli, T., Koskimies, P. & Hyypia, T. (1995). Molecular comparison of coxsackie A virus serotypes. Virology 212, 30-8.

Romero, J. R. (1999). Reverse-transcription polymerase chain reaction detection of the enteroviruses. Arch Pathol Lab Med 123, 1161-9.

Rotbart, H. A. (1995). Enteroviral infections of the central nervous system. Clinical Infectious Diseases 20, 971-981.

Rueckert, R. R. (1996). Picornaviridae: The Viruses and Their Replication. In Fields Virology, third edition, pp. 609-654. Edited by D. M. K. B. N. Fields, P. M. Howley, R. M. Channock, J. L. Melnick, T. P. Monath, B. Roizman, and S. E. Straus. Philadelphia: Lippincott-Raven Publishers.

Sambrook, J., Fritsch, E. F. & Maniatis, T. (1989). Molecular cloning: a laboratory manual. Cold Spring Harbor Laboratory Press.

Strimmer, K. & von Haeseler, A. (1996). Quartet Puzzling: A quartet maximum-likelihood method for reconstructing tree topologies. Mol. Biol. Evol. 13, 964-969.

Thompson, J. D., Higgins, D. G. & Gibson, T. J. (1994). CLUSTAL W: improving the sensitivity of progressive multiple sequence alignment through sequence weighting, position-specific gap penalties and weight matrix choice. Nucleic Acids Research 22, 4673-80.

Zhang, G., Haydon, D. T., Knowles, N. J. & McCauley, J. W. (1999). Molecular evolution of swine vesicular disease virus. J Gen Virol 80, 639-51.

Zimmermann, H., Eggers, H. J. & Nelsen-Salz, B. (1996). Molecular cloning and sequence determination of the complete genome of the virulent echovirus 9 strain barty. Virus Genes 12, 149-54.

Zimmermann, H., Eggers, H. J. & Nelsen-Salz, B. (1997). Cell attachment and mouse virulence of echovirus 9 correlate with an RGD motif in the capsid protein VP1. Virology 233, 149-56.

TABLE 1
Primers used for RT-PCR and sequencing of Human Enteroviruses

| | | | | Genomic |
|---|---|---|---|---|
| Primer | Sequence* | Polarity | Gene | location† |
| EUC2 | 5′ TTT GCA CTT GAA CTG TAT GTA 3′ | Antisense | 2C | 4454-4474 |
| EUC2a | 5′ GGT TCA ATA CGG CAT TTG GA 3′ | Antisense | 2C | 4469-4488 |
| EUC2b | 5′ GGT TCA ATA CGG TGT TTG CT 3′ | Antisense | 2C | 4469-4488 |
| EUG3a | 5′ TGG CAA ACT TCC WCC AAC CC 3′ | Sense | VP1 | 3002-3021 |
| EUG3b | 5′ TGG CAA ACA TCT TCM AAT CC 3′ | Sense | VP1 | 3002-3021 |
| EUG3c | 5′ TGG CAG ACT TCA ACH AAC CC 3′ | Sense | VP1 | 3002-3021 |

*Sequences are given using standard nucleotide ambiguity codes
†PV1 Mahoney numbering

TABLE 2
Details of enteroviruses isolates used in this study

| No. | Isolate name* | Serotype | Geographic location | Sample origin | Pathology | Accession no.† |
|---|---|---|---|---|---|---|
| 1 | RO-14/5/70 | CB5 | Romania | blood | meningitis | AJ279151 |
| 2 | RO-79/2/71 | E5 | Romania | stool | meningitis | AJ279152 |
| 3 | RO-29/6/72 | E11 | Romania | naso-pharyngeal secretion | meningitis | AJ279153 |
| 4 | RO-1/9/72 | E9 | Romania | spinal cord | encephalitis | AJ279154 |
| 5 | RO-86/1/73 | CB6 | Romania | stool | spinal paralysis | AJ279155 |
| 6 | RO-78/3/74 | E12 | Romania | stool | facial paralysis | AJ279156 |
| 7 | RO-122/1/74 | E1 | Romania | stool | spinal paralysis | AJ279157 |
| 8 | RO-98/1/74 | CB1 | Romania | stool | spinal paralysis | AJ279158 |
| 9 | RO-112/1/78 | E8 | Romania | stool | meningitis | AJ279159 |
| 10 | RO-24/9/79 | E6 | Romania | spinal cord | meningitis | AJ279160 |
| 11 | RO-81/1/79 | E14 | Romania | stool | spinal paralysis | AJ279161 |
| 12 | RO-28/12/79 | E6 | Romania | cortex | bronchopneumonia | AJ279162 |
| 13 | RO-609/4/80 | CA9 | Romania | cerebro-spinal fluid | meningitis | AJ279163 |
| 14 | RO-434/2/81 | E7 | Romania | stool | spinal paralysis | AJ279164 |
| 15 | RO-543/1/81 | E11 | Romania | stool | spinal paralysis | AJ279165 |
| 16 | RO-116/6/82 | E9 | Romania | naso-pharyngeal secretion | rachialgia | AJ279166 |
| 17 | RO-104/1/82 | E7 | Romania | stool | spinal paralysis | AJ279167 |
| 18 | RO-69/1/86 | CB4 | Romania | stool | hemiparesis | AJ279168 |
| 19 | NL-16271/87 | E11 | The Netherlands | stool | meningitis | AJ279169 |
| 20 | NL-2463/88 | E11 | The Netherlands | stool | gastro-intestinal disorder | AJ279170 |
| 21 | NL-9691/88 | E11 | The Netherlands | stool | pharyngitis | AJ279171 |
| 22 | NL-8120/88 | E11 | The Netherlands | stool | respiratory infection | AJ279172 |
| 23 | RO-69/1/89 | CB3 | Romania | stool | spinal paralysis | AJ279173 |
| 24 | FR-2689/91 | E30 | France | stool | meningeal syndrome | AJ279174 |
| 25 | RO-38/3/91 | CA9 | Romania | stool | spinal paralysis | AJ279175 |
| 26 | FR-1477/93 | E25 | France | stool | not known | AJ279176 |
| 27 | GR-KYR/94 | CA16 | Greece | vesicle | hand, foot, and mouth disease | AJ279177 |
| 28 | GR-CHR/94 | CA16 | Greece | vesicle | hand, foot, and mouth disease | AJ279178 |
| 29 | MG-354/94 | ut‡ | Madagascar | stool | healthy | AJ279179 |
| 30 | MG-356/94 | ut | Madagascar | stool | healthy | AJ279180 |
| 31 | MG-404/94 | ut | Madagascar | stool | healthy | AJ279181 |
| 32 | MG-448/94 | ut | Madagascar | stool | healthy | AJ279182 |
| 33 | MG-451/94 | CB | Madagascar | stool | healthy | AJ279183 |
| 34 | MG-423/94 | ut | Madagascar | stool | healthy | AJ279184 |
| 35 | MG-498/94 | ut | Madagascar | stool | healthy | AJ279185 |
| 36 | FR-1254/95 | CB | France | throat | not known | AJ279186 |
| 37 | FR-2272/95 | CB | France | throat | not known | AJ279187 |
| 38 | RO-141/2/95 | E7 | Romania | stool | facial paralysis | AJ279188 |
| 39 | RO-123/1/95 | CB3 | Romania | stool | meningo-encephalitis | AJ279189 |
| 40 | FR-3574/96 | E30 | France | cerebro-spinal fluid | meningitis | AJ279190 |
| 41 | FR-1829/96 | E30 | France | cerebro-spinal fluid | meningitis | AJ279191 |
| 42 | FR-2479/96 | E30 | France | cerebro-spinal fluid | meningitis | AJ279192 |
| 43 | MG-41094/97 | E11 | Madagascar | stool | acute flaccid paralysis | AJ279193 |
| 44 | BF-01/98 | E33 | Burkina Faso | stool | acute flaccid paralysis contact | AJ279194 |
| 45. | MG-44381/98 | E7 | Madagascar | stool | acute flaccid paralysis | AJ279195 |

*Isolates were described by the country of origin (two-letter code), laboratory identifier and year of isolation (two-figure code)
†Sequences have been deposited in the EMBL database
‡Strains “untypeable” with RIVM pools

TABLE 3
Correspondence between partial VP1 sequencing and seroneutralisation

| | | | Highest-score prototype* | | | Second-highest-score prototype | | | | |
|---|---|---|---|---|---|---|---|---|---|---|
| | | | | sequence | identity | | sequence | | identity | Delta Score† |
| | Isolate | Serotype | Type | % nt | % aa | Type | % nt | Type | % aa | % nt |
| 1 | RO-14/5/70 | CB5 | SVDV | 89.2 | 95.5 | CB5 | 78.5 | CB5 | 95.5 | 10.7 |
| 2 | RO-79/2/71 | E5 | E5 | 82.7 | 95.0 | E16 | 71.8 | E31 | 80.3 | 10.9 |
| 3 | RO-29/6/72 | E11 | E11 | 80.3 | 94.2 | E19 | 72.0 | E19 | 83.4 | 08.3 |
| 4 | RO-1/9/72 | E9 | E9 | 89.4 | 94.3 | E16 | 74.3 | E16 | 78.2 | 15.1 |
| 5 | RO-86/1/73 | CB6 | CB6 | 79.4 | 93.7 | E21 | 70.8 | CB3 | 72.3 | 08.6 |
| 6 | RO-78/3/74 | E12 | E12 | 81.7 | 95.9 | E11 | 70.0 | E11 | 73.5 | 11.7 |
| 7 | RO-122/1/74 | E1 | E1 | 80.5 | 97.3 | E8 | 77.0 | E8 | 92.6 | 03.5 |
| 8 | RO-98/1/74 | CB1 | CB1 | 81.4 | 95.3 | CB3 | 71.4 | CB3 | 83.1 | 10.0 |
| 9 | RO-112/1/78 | E8 | E8 | 83.4 | 98.2 | E1 | 76.3 | E1 | 94.6 | 07.1 |
| 10 | RO-24/9/79 | E6 | E6 | 81.4 | 94.8 | E24 | 73.6 | E29 | 77.4 | 07.8 |
| 11 | RO-81/1/79 | E14 | E14 | 81.4 | 92.0 | E2 | 70.6 | E31 | 78.9 | 10.8 |
| 12 | RO-28/12/79 | E6 | E6 | 81.3 | 94.0 | E24 | 73.5 | E29 | 77.3 | 07.8 |
| 13 | RO-609/4/80 | CA9 | CA9 | 80.3 | 94.6 | E11 | 70.6 | E9 | 70.4 | 09.7 |
| 14 | RO-434/2/81 | E7 | E7 | 79.1 | 95.8 | E11 | 68.9 | E11 | 74.1 | 10.2 |
| 15 | RO-543/1/81 | E11 | E11 | 81.2 | 93.3 | E19 | 76.5 | E19 | 83.4 | 04.7 |
| 16 | RO-116/6/82 | E9 | E9 | 83.9 | 93.4 | E5 | 74.8 | E16 | 78.0 | 09.1 |
| 17 | RO-104/1/82 | E7 | E7 | 79.4 | 96.6 | E11 | 68.7 | E11 | 74.7 | 10.7 |
| 18 | RO-69/1/86 | CB4 | CB4 | 84.9 | 96.3 | CB3 | 71.5 | CB2 | 77.1 | 13.4 |
| 19 | NL-16271/87 | E11 | E11 | 80.5 | 94.2 | E19 | 75.2 | E19 | 84.2 | 05.3 |
| 20 | NL-2463/88 | E11 | E11 | 79.8 | 93.3 | E19 | 75.7 | E19 | 84.2 | 04.1 |
| 21 | NL-9691/88 | E11 | E11 | 81.4 | 95.0 | E19 | 75.2 | E19 | 84.8 | 06.2 |
| 22 | NL-8120/88 | E11 | E11 | 81.7 | 94.2 | E19 | 75.7 | E19 | 83.4 | 06.0 |
| 23 | RO-69/1/89 | CB3 | CB3 | 81.0 | 97.2 | E13 | 72.5 | CB1 | 81.6 | 08.5 |
| 24 | FR-2689/91 | E30 | E30 | 88.4 | 96.5 | E21 | 69.0 | E21 | 84.0 | 19.4 |
| 25 | RO-38/3/91 | CA9 | CA9 | 82.4 | 93.8 | E9 | 67.9 | E9 | 70.4 | 14.5 |
| 26 | FR-1477/93 | E25 | E25 | 79.6 | 92.5 | E21 | 72.3 | E21 | 83.4 | 07.3 |
| 27 | GR-KYR/94 | CA16 | CA16 | 80.8 | 96.2 | EV71 | 70.0 | EV71 | 76.8 | 10.8 |
| 28 | GR-CHR/94 | CA16 | CA16 | 78.9 | 93.8 | EV71 | 68.9 | EV71 | 73.4 | 10.0 |
| 29 | MG-354/94 | ut | CA13 | 77.9 | 90.6 | CA18 | 76.3 | CA18 | 89.8 | 01.6 |
| 30 | MG-356/94 | ut | CA13 | 77.4 | 89.8 | CA18 | 75.4 | CA18 | 88.9 | 02.0 |
| 31 | MG-404/94 | ut | CA13 | 80.2 | 92.3 | CA18 | 78.0 | CA18 | 91.5 | 02.2 |
| 32 | MG-448/94 | ut | CA20 | 74.8 | 93.3 | CA17 | 71.9 | CA18 | 82.5 | 02.9 |
| 33 | MG-451/94 | CB | CB6 | 78.1 | 92.0 | CB3 | 69.5 | E11 | 70.7 | 08.6 |
| 34 | MG-423/94 | ut | CA20 | 75.0 | 90.6 | CA13 | 72.3 | CA13 | 80.3 | 02.7 |
| 35 | MG-498/94 | ut | CA13 | 78.7 | 93.2 | CA18 | 77.3 | CA18 | 92.4 | 01.4 |
| 36 | FR-1254/95 | CB | CB1 | 81.2 | 98.1 | CB3 | 73.6 | CB3 | 84.2 | 07.6 |
| 37 | FR-2272/95 | CB | CB1 | 81.3 | 96.4 | CB3 | 72.4 | CB3 | 84.6 | 08.9 |
| 38 | RO-141/2/95 | E7 | E7 | 78.7 | 96.6 | E11 | 68.7 | E11 | 74.7 | 10.0 |
| 39 | RO-123/1/95 | CB3 | CB3 | 81.1 | 97.9 | CB1 | 71.7 | CB1 | 80.5 | 09.4 |
| 40 | FR-3574/96 | E30 | E30 | 88.5 | 97.5 | E21 | 70.9 | E21 | 84.6 | 17.6 |
| 41 | FR-1829/96 | E30 | E30 | 89.1 | 97.5 | E21 | 70.3 | E21 | 84.6 | 18.8 |
| 42 | FR-2479/96 | E30 | E30 | 89.1 | 97.5 | E21 | 70.3 | E21 | 84.6 | 18.8 |
| 43 | MG-41094/97 | E11 | E11 | 79.3 | 91.8 | E19 | 71.5 | E19 | 83.7 | 07.8 |
| 44 | BF-01/98 | E33 | E33 | 78.8 | 95.5 | E4 | 69.0 | E19 | 77.9 | 09.8 |
| 45 | MG-44381/98 | E7 | E7 | 77.5 | 95.0 | E2 | 72.4 | E11 | 76.2 | 05.1 |

*nt, nucleotide; aa, amino acid
†Delta score is the difference in percentage nucleotide sequence identity between the highest- and the second-highest-score prototype

1. A purified polynucleotide selected from the group consisting of SEQ ID NO:1, SEQ ID NO:2, SEQ ID NO:3, SEQ ID NO:4, SEQ ID NO:5 and SEQ ID NO:6.

2. The purified polynucleotide of claim 1 which is SEQ ID NO:1.

3. The purified polynucleotide of claim 1 which is SEQ ID NO:2.

4. The purified polynucleotide of claim 1 which is SEQ ID NO:3.

5. The purified polynucleotide of claim 1 which is SEQ ID NO:4.

6. The purified polynucleotide of claim 1 which is SEQ ID NO:5.

7. The purified polynucleotide of claim 1 which is SEQ ID NO:6.

8. A purified polynucleotide selected from the group consisting of SEQ ID NOS:7-51.

9. A purified polynucleotide consisting of a sequence which stringently hybridizes to one or more of the polynucleotides of claim 1, wherein said polynucleotide is from 15 to 25 nucleotides in length.

10. A purified polynucleotide consisting of a sequence which stringently hybridizes to one or more of the polynucleotides of claim 8.

11. A method of detecting an Enterovirus in a sample comprising
contacting a polynucleotide region of a VP1 -2C genes of said enterovirus with amplification primers;
amplifying said polynucleotide region of the VP1-2C gene; and
detecting the presence of an amplified polynucleotide, wherein the presence of the amplified product is indicative of the presence of the Enterovirus in the sample.

12. The method of claim 11, wherein the amplified polynucleotide comprises at least one sequence selected from the group consisting of SEQ ID NOS:7-51 or a sequence which stringently hybridizes to one or more of the sequences SEQ ID NOS:7-51.

13. The method of claim 11, wherein said amplification primers are selected from the group consisting of SEQ ID NO:1, SEQ ID NO:2, SEQ ID NO:3, SEQ ID NO:4, SEQ ID NO:5 and SEQ ID NO:6.

14. The method of claim 11, wherein said amplifying is RT-PCR.

15. The method of claim 11, wherein said detecting comprises hybridization of said amplified product with a probe corresponding to the Enterovirus VP1-2C genes or a fragment thereof.

16. The method of claim 15, wherein said probe is the 3′ third of the VP1 gene or a fragment thereof.

17. The method of claim 15, wherein said probe is selected from the group consisting of SEQ ID NOS:7-51 and fragments thereof, sequences which stringently hybridize with SEQ ID NOS:7-51 and fragments thereof.

18. The method of claim 15, wherein said detecting comprises serotyping the enterovirus with a probe specific for an enterovirus serotype.

19. A purified polynucleotide hybridizing under stringent conditions with the amplified polynucleotide of the VP1-2C gene produced by the method of claim 11.

20. A purified polypeptide encoded by the isolated polynucleotide of claim 19.

21. A method of detecting the presence of an Enterovirus in a sample, comprising:
contacting an purified and purified nucleic acid comprising (a) a polynucleotide sequence contained in the VP1-2C genes of said Enterovirus with the sample containing (b) an enterovirus; detecting the presence of hybridization between (a) and (b), wherein the presence of hybridization between (a) and (b) is indicative of the presence of the enterovirus in the sample.

22. The method of claim 21, wherein purified and purified nucleic acid is SEQ ID NO:7-51 or fragments thereof or sequences which stringently hybridize to one or more of the sequences SEQ ID NOS:7-51 or fragments thereof.

23. A method of detecting the presence of an Enterovirus in an Enterovirus sample, comprising
contacting a polynucleotide region of VP1-2C genes of said enterovirus with amplification primers;
amplifying said polynucleotide region of the VP1-2C genes;
sequencing said amplified polynucleotide region; and
comparing the sequence of said amplified polynucleotide region with known sequences of enteroviruses of various serotypes;
wherein an identity score of at least 75% indicates that the two compared sequences are from enteroviruses of the same phenotype.

24. The method of claim 23, wherein said amplification primers are selected from the group consisting of SEQ ID NO:1-6.

25. The method of claim 23, wherein said amplifying is RT-PCR.

26. A kit comprising one or more of the polynucleotides of claim 1.

27. A kit comprising one or more of the polynucleotides of claim 8 or fragments thereof or the purified polynucleotides which stringently hybridize with SEQ ID NOS:7-51 or a fragment thereof.

28. The kit of claim 26, further comprising one or more reagents necessary for RNA and/or DNA amplification.

29. The kit of claim 28, further comprising one or more reagents necessary for RNA and/or DNA amplification.

30. The kit of claim 26, further comprising one or more reagents necessary for RNA and/or DNA hybridization.

31. The kit of claim 28, further comprising one or more reagents necessary for RNA and/or DNA hybridization.

32. A purified polynucleotide hybridizing under stringent conditions with the amplified polynucleotide of a VP1-2C gene produced by the method of claim 11 and the two amplification primers.

33. A purified polypeptide encoded by the purified polynucleotide of claim 32.


SEQUENCE LISTING (96 sequences)

SEQ ID NO: 1 - DNA, 21 bp, ARTIFICIAL SEQUENCE
  SYNTHETIC DNA
tttgcacttgaactgtatgta

SEQ ID NO: 2 - DNA, 20 bp, ARTIFICIAL SEQUENCE
  SYNTHETIC DNA
ggttcaatacggcatttgga

SEQ ID NO: 3 - DNA, 20 bp, ARTIFICIAL SEQUENCE
  SYNTHETIC DNA
ggttcaatacggtgtttgct

SEQ ID NO: 4 - DNA, 20 bp, ARTIFICIAL SEQUENCE
  SYNTHETIC DNA
tggcaaacttccwccaaccc

SEQ ID NO: 5 - DNA, 20 bp, ARTIFICIAL SEQUENCE
  SYNTHETIC DNA
tggcaaacatcttcmaatcc

SEQ ID NO: 6 - DNA, 20 bp, ARTIFICIAL SEQUENCE
  SYNTHETIC DNA
tggcagacttcaachaaccc

SEQ ID NO: 7 - DNA, 783 bp, Coxsackievirus B
  CDS (1)..(783)
acggaaggaagtgcaccacctcgaatgtcaataccattcattagcataggcaatgcatacagcatgttctatgacgggtgggccaggtttgataaacaagggacatatggcatcaacacactaaacaacatggggacactgtatatgagacatgtgaatgacggtagccccggtcccatcgtgagtacggtgcgaatttacttcaaaccgaagcacgttaagacgtgggtcccaagaccgcctaggttgtgtcaatatcagaaggcaggtaacgtgaattttgaacccactggtgtgaccgagggtaggacagacataaccaccatgcaaactactggcgccttcgggcagcagtctggtgctgtatacgtcggcaactacagagtggtgaatagacatcttgcaacacacatggactggcaaaattgtgtttgggaggattacaacagagaccttctagttagcaccaccacagcccacgggtgtgacaccatagctagatgccaatgcacgacaggagtatacttctgtcaatccagaaacaaacattatccggtgagttttgaagggccagggcttgtggaagttcaagagagtgagtattaccccagaaggttccagtcccatgtactcctagccgccggattctccgagcccggtgactgcggtggcatcttaaggtgtgatcacggtgtcatcggagtcgtgaccatgggtggtgaagggattgtcggctttgctgatataagagaccttctatggttggaggacgatgccatggag

SEQ ID NO: 8 - protein, 261 aa, Coxsackievirus B
TEGSAPPRMSIPFISIGNAYSMFYDGWARFDKQGTYGINTLNNMGTLYMRHVNDGSPGPIVSTVRIYFKPKHVKTWVPRPPRLCQYQKAGNVNFEPTGVTEGRTDITTMQTTGAFGQQSGAVYVGNYRVVNRHLATHMDWQNCVWEDYNRDLLVSTTTAHGCDTIARCQCTTGVYFCQSRNKHYPVSFEGPGLVEVQESEYYPRRFQSHVLLAAGFSEPGDCGGILRCDHGVIGVVTMGGEGIVGFADIRDLLWLEDDAME

SEQ ID NO: 9 - DNA, 633 bp, Echovirus
  CDS (1)..(633)
gtattttggactgaaggaaacgcgcctgccagaatgtctatacctttcattagcattggcaacgcatacagtagtttttatgatggatggtcacacttcacccaaaagggaatctatggatacaatacactgaataagatgggtcagctgtttgttagacatgtgaacaaagagactccgacaccggttaccagtaccataagggtttacttcaagccaaagcacatcagggcttgggtccctagacccccacgattgtgcccctacgtaaacaagacaaatgtgaatttcaacaccacgcaggtgacaaaaccaaggaacgatctcaatgacgtgccaaagtccgatcacaatgtgcgtacgtacggggcctttggacaacaatccggcgccgtgtacgtgggcaactacagactggtgaatagacacctcgccacacatacagactggcagagttgtgtttgggaagattataacagagaccttctcgtgtgtactaccacagcacacggatgtgacaccattgccagatgccagtgcacaactggagtgtatttctgccaatccaggaataagcactacccggttagttttgaaggaccgggcatagtggaagttcaagaaagtgaatat

SEQ ID NO: 10 - protein, 211 aa, Echovirus
VFWTEGNAPARMSIPFISIGNAYSSFYDGWSHFTQKGIYGYNTLNKMGQLFVRHVNKETPTPVTSTIRVYFKPKHIRAWVPRPPRLCPYVNKTNVNFNTTQVTKPRNDLNDVPKSDHNVRTYGAFGQQSGAVYVGNYRLVNRHLATHTDWQSCVWEDYNRDLLVCTTTAHGCDTIARCQCTTGVYFCQSRNKHYPVSFEGPGIVEVQESEY

SEQ ID NO: 11 - DNA, 732 bp, Echovirus
  CDS (1)..(732)
tggactgaggggaatgcaccacctagaatgtccatccccttcattagcatcggtaatgcctacagtaatttctatgatggttggtcacacttttcccaaaatggtgtgtacggatacaacacgctcaatcatatgggccaaatttacgttagacacttgaacggatcctctccactacccatgacgagtacagttaggatgtacttcaagccaaaacatgtgaaagtgtgggtcccgcgcccgcctagattgtgccagtacaaaaacgcatcaactgtcaacttcacacccaccaacattactgaaaagaggaaaagcattaattacataccagagacggttaagccggatgtttcgacgtacggagcgttcggtcagcaatcaggagcaatttacgtgggtaattatagggtggtgaatagacatctagccacccacaccgactggcaaaattgtgtgtgggataactacaacagagatctattagtcagcactactactgcgcatgggtgcgacaccatcgccagatgccagtgcacaacgggcgtgtatttttgtgcttcgaaaaacaagcattacccgatcagcttcgaaggaccaggtctggtggaagtacaggaaagtgagtactacccacgaaggtatcagtcccacgttttattggcagcagggttttcagagcccggagattgtggtggaatcctgaggtgcgaacatggtgtcattggactc

SEQ ID NO: 12 - protein, 244 aa, Echovirus
WTEGNAPPRMSIPFISIGNAYSNFYDGWSHFSQNGVYGYNTLNHMGQIYVRHLNGSSPLPMTSTVRMYFKPKHVKVWVPRPPRLCQYKNASTVNFTPTNITEKRKSINYIPETVKPDVSTYGAFGQQSGAIYVGNYRVVNRHLATHTDWQNCVWDNYNRDLLVSTTTAHGCDTIARCQCTTGVYFCASKNKHYPISFEGPGLVEVQESEYYPRRYQSHVLLAAGFSEPGDCGGILRCEHGVIGL

SEQ ID NO: 13 - DNA, 663 bp, Echovirus
  CDS (1)..(663)
tggactgaaggcaatgctccacctaggatgtccattccgttcattagtataggcaatgcctatagtagcttttatgatgggtggtcacattttgatagcaaaggtgcctacggtttcaacactttgaacaaaatgggacacatctactgcaggcacgtgaataaagagacacccgctgaggtcaccagttatattagaatttatttcaagcccaagcacattagagcgtgggttccaagaccaccgcgactttgtcagtacaaaaataaggcaaacgtcaactttgacgccacaccagtcactgatacgcgtgacaccatcaacactgtccccgtatcaagtcatgggggtgtacgccgtggtgatttggctgcactgagcacacgtggtgcttttggccaacaatccggtgctatatacgtgggcaactacagggtggttaacaggcacttagccacacgcactgattggcagaactgcgtttgggaagattataacagggatcttctagtgagcacaacaaccgcccatggatgcgacacaatagccagatgccagtgcaaaacaggcgtatacttctgccaatccaagggcaagcactacccggttagtttcgagggtccagggctggtagaggtccagaagagtgaatactaccccagg

SEQ ID NO: 14 - protein, 221 aa, Echovirus
WTEGNAPPRMSIPFISIGNAYSSFYDGWSHFDSKGAYGFNTLNKMGHIYCRHVNKETPAEVTSYIRIYFKPKHIRAWVPRPPRLCQYKNKANVNFDATPVTDTRDTINTVPVSSHGGVRRGDLAALSTRGAFGQQSGAIYVGNYRVVNRHLATRTDWQNCVWEDYNRDLLVSTTTAHGCDTIARCQCKTGVYFCQSKGKHYPVSFEGPGLVEVQKSEYYPR

SEQ ID NO: 15 - DNA, 777 bp, Coxsackievirus B
  CDS (1)..(777)
actgagggtaacgcaccacctcgagtgtctgtgcccttcatgagtattggaaatgcctatagcaccttctatgatggctggtctgatttttcatcaaagggagtgtatggcttgaacactctgaataacatgggaacattgtatattcggcacgtcaatgggcctaacccagtgcctatcaccagcaccgtgcgtatctattttaaaccaaagcatgttaaggcatgggtgccacgaccaccaagattatgccaatacaacacatcaaggcaagtcaacttcagtgtgacgggagtaacagaatccagggagaatataacaactatgaaaaccactggggcttttggtcaacagtctggagcagcgtatgtgggcaactataggatagttaacagacacctggcaactcacaccgactggcagagttgtgtatgggaagactacaacagggaccttcttgtaagcaccaccacagcacacgggtgtgacaccatagcaaggtgtcgttgcaactctggtgtgtacttctgtgcttctaagaacaaacactacccggtgagtttcgaggggccaggcttggtggaagtccaggagagtgagtactaccctaagagataccagtcccatgttctcctagcagtcgggttctccgaaccaggtgactgtggtggcatcttaaggtgtgaacacggtgtcatcggccttgtcactatgggtggtgaaagtgttgtggggttcacagatatccgcgacctgctgtggattgaggacgacgct

SEQ ID NO: 16 - protein, 259 aa, Coxsackievirus B
TEGNAPPRVSVPFMSIGNAYSTFYDGWSDFSSKGVYGLNTLNNMGTLYIRHVNGPNPVPITSTVRIYFKPKHVKAWVPRPPRLCQYNTSRQVNFSVTGVTESRENITTMKTTGAFGQQSGAAYVGNYRIVNRHLATHTDWQSCVWEDYNRDLLVSTTTAHGCDTIARCRCNSGVYFCASKNKHYPVSFEGPGLVEVQESEYYPKRYQSHVLLAVGFSEPGDCGGILRCEHGVIGLVTMGGESVVGFTDIRDLLWIEDDA

SEQ ID NO: 17 - DNA, 744 bp, Echovirus
  CDS (1)..(744)
tggacggagggcaacgcacctgcgagaatgtccatcccattcatcagcattggtaatgcgtacagcaatttttatgatgggtggtcacactttacacaagatggggtgtatgggttcaactctctcaataacatgggctctatatacattagacatgtcaacgagcagagtccccatgctatcacgagcactgtgagagtgtacttcaaacccaaacacgtgagggcatgggtcccaaggccaccaaggttatgtgcatatgaaaaatctagcaacgtaaacttcaaacccacagatgtgaccactacgcgctcatccatcacggaagtccccagtcttaggccatcggtacaaaacaccggggctttcggacaacaatccggagcagtttacgttggaaactatagagttgttaacaggcacttagccacccacaatgattggcaaaattgcgtgtgggaagattacaatagagatcttcttataagcacaacaactgctcacgggtgtgacaccatagccagatgccggtgcacaacaggagtgtatttctgtcaatccaaaaacaaacactacccagttagttttgaagggccagggttagtggaagttcaagagagtgagtactatcctaaaaggtatcaatcccacgtgctacttgctgcaggtttctctgagccaggcgactgtggtggaatcttgaggtgtgaacatggcgtcatcggtattgtgaccatgggtggt

SEQ ID NO: 18 - protein, 248 aa, Echovirus
WTEGNAPARMSIPFISIGNAYSNFYDGWSHFTQDGVYGFNSLNNMGSIYIRHVNEQSPHAITSTVRVYFKPKHVRAWVPRPPRLCAYEKSSNVNFKPTDVTTTRSSITEVPSLRPSVQNTGAFGQQSGAVYVGNYRVVNRHLATHNDWQNCVWEDYNRDLLISTTTAHGCDTIARCRCTTGVYFCQSKNKHYPVSFEGPGLVEVQESEYYPKRYQSHVLLAAGFSEPGDCGGILRCEHGVIGIVTMGG

SEQ ID NO: 19 - DNA, 654 bp, Echovirus
  CDS (1)..(654)
actgaggggaatgcaccagctcgcatgtcaattccattcatcagcataggcaatgcttacagtaacttctatgatggatggtcacacttttcacaaaccggtgtctatggttttactacactgaataatatgggtcaactgttcttcagacacgttaacaagcctaacccggctgccattaccagtgtagcacgcatatatttcaagccaaaacacgtgcgtgcttgggtgcccaggccaccacgcctgtgcccgtatatcaatagcaccaatgtcaatttcaaacctaagccagtgacaaaggtgcggaataacataataacaacaggagcatttggacagcagtctggtgcagtttacgtaggaaactacagggtagtgaacaggcatctcgctacacataatgattggcagaactgtgtgtgggaagattacagtagagaccttctagtgagcaccaccaccgcccacgggtgcgacactatagccagatgccaatgtacgacaggtgtatacttctgtgcctcgaagaacaagcactaccatattacttttgaggggccaggtctagtggaagttcaagagagtgaatactaccccaagaggtttcaatcacacgtcttactagctacagggttctcggaaccaggagac

SEQ ID NO: 20 - protein, 218 aa, Echovirus
TEGNAPARMSIPFISIGNAYSNFYDGWSHFSQTGVYGFTTLNNMGQLFFRHVNKPNPAAITSVARIYFKPKHVRAWVPRPPRLCPYINSTNVNFKPKPVTKVRNNIITTGAFGQQSGAVYVGNYRVVNRHLATHNDWQNCVWEDYSRDLLVSTTTAHGCDTIARCQCTTGVYFCASKNKHYHITFEGPGLVEVQESEYYPKRFQSHVLLATGFSEPGD

SEQ ID NO: 21 - DNA, 777 bp, Coxsackievirus B
  CDS (1)..(777)
acggaggggaatgctccaccaaggatgtcaattccatttattagcattggcaatgcgtatagctgtttttatgatgggtggacacagttctcaagaaacggggtctatgggattaacactctaaacaacatgggcactttatacatgcggcatgtcaacgaagcaggacagggcccaattaagagtactgttagaatatatttcaaacctaagcatgtgaaggcgtgggtgccccgcccgcctaggctgtgtcagtatgagaaacaaaagaatgtcaattttaaacccacaggagtaaccacacataggctggacattgtaacaacaggcgcttttggtcagcaatctggcgcaatatacgtaggaaactacagagtggttaacagacacttagccacgcacaaagattgggagagctgcgtgtgggaagactacaacagagacctcctagtgagcaccaccaccgcccacgggtgtgacaccatagctagatgccaatgtacgacaggtgtgtacttctgtgcctccaagaacaagcactaccccgtcgcttttgaggggccaggtctagtggaagttcaggaaagcgaatactacccaaagagatatcagtcccatgtgttgctcgctgcgggattttcagagccaggcgattgtggaggtatcctgaggtgcgagcacggagtcattggacttgtgaccatggggggtgaaggcatcgtcggctttgctgatgtgcgcgacatcctatggttggaagatgacgccatggaacag

SEQ ID NO: 22 - protein, 259 aa, Coxsackievirus B
TEGNAPPRMSIPFISIGNAYSCFYDGWTQFSRNGVYGINTLNNMGTLYMRHVNEAGQGPIKSTVRIYFKPKHVKAWVPRPPRLCQYEKQKNVNFKPTGVTTHRLDIVTTGAFGQQSGAIYVGNYRVVNRHLATHKDWESCVWEDYNRDLLVSTTTAHGCDTIARCQCTTGVYFCASKNKHYPVAFEGPGLVEVQESEYYPKRYQSHVLLAAGFSEPGDCGGILRCEHGVIGLVTMGGEGIVGFADVRDILWLEDDAMEQ

SEQ ID NO: 23 - DNA, 735 bp, Echovirus
  CDS (1)..(735)
agcatattctggacagaggggaatgcacctgctaggatgtccatacccttcattagcatcggaaatgcatacagtaacttctatgacgggtggtcacacttctcacagaacggggtttacggtttcaccactttgaacaacatgggccagctattcttcagacacgttaacaagcccaatccggcaacaatcaccagcgtagctcgcatttacttcaagcccaaacacgtgagagcctgggtgcctagaccacctcggttgtgcccctatatcaatagtggcaatgtaaattttgacccaaaacccgtgacggaagtcagaagcagcatcatcaccaccggggccttcgggcaacagtcaggggcggtttacgtgggaaattacagggtggtgaatagacacctcgccacgcgcaatgattggttaagttgcgtgtgggaagattacaacagagacctcctagtgagcaccaccacagcgcatggctgcgacactatagccaggtgccgttgcaccacgggtgtatacttttgcgcgtccaaaaacaaacactacccggtctcctttgaagggccaggcttagtggaagtccaggagagtgaatactatcccaaaagatatcaatcccatgttcttttagcggccgggttctccgacccgggcgattgtggtggtatcttgaggtgcgagcacggtgtcatcggtctcgttaccatgggtggtgaaggcgttgta

SEQ ID NO: 24 - protein, 245 aa, Echovirus
SIFWTEGNAPARMSIPFISIGNAYSNFYDGWSHFSQNGVYGFTTLNNMGQLFFRHVNKPNPATITSVARIYFKPKHVRAWVPRPPRLCPYINSGNVNFDPKPVTEVRSSIITTGAFGQQSGAVYVGNYRVVNRHLATRNDWLSCVWEDYNRDLLVSTTTAHGCDTIARCRCTTGVYFCASKNKHYPVSFEGPGLVEVQESEYYPKRYQSHVLLAAGFSDPGDCGGILRCEHGVIGLVTMGGEGVV

SEQ ID NO: 25 - DNA, 720 bp, Echovirus
  CDS (1)..(720)
tggattgagggaaacgccccccccagaatgtccatcccttttatgagtgtgggaaacgcatatagcaacttctacgatggatggtctcacttttcgcaaacaggtgtgtacggtttcaacactctcaacaacatggggaagttatatttcagacatgtgaacgacaagacaattagcccaatcacaagcaaagtccgcatctacttcaaaccaaaacacgtgaaagcttgggtaccccgcccacctagactgtgtgagtacacgcacaaagacaatgtggattttgaacccaaaggcgttaccacatcccgcacccaactaacaattagcaactccacacacatggaaaattatggtgcctttggacaacaatctggagccgtctacgtgggcaattatagactagtaaacagacacctagccacgcacaatgattggcagaactgtgtatgggaagactacaatagagaccttttagttagcaccaccacagcccatggatgtgacactattgctaggtgccaatgcaccactggtgtatacttttgtgcttccaaaaacaagcactatcccgtggcatttgagggacctggtctagtggaagtgcaggagagcgagtattaccctaaaagatatcagtctcatgttcttcttgccgcagggttctccgagccaggggattgtggtggcatccttaggtgtgaacatggtgtcataggc

SEQ ID NO: 26 - protein, 240 aa, Echovirus
WIEGNAPPRMSIPFMSVGNAYSNFYDGWSHFSQTGVYGFNTLNNMGKLYFRHVNDKTISPITSKVRIYFKPKHVKAWVPRPPRLCEYTHKDNVDFEPKGVTTSRTQLTISNSTHMENYGAFGQQSGAVYVGNYRLVNRHLATHNDWQNCVWEDYNRDLLVSTTTAHGCDTIARCQCTTGVYFCASKNKHYPVAFEGPGLVEVQESEYYPKRYQSHVLLAAGFSEPGDCGGILRCEHGVIG

SEQ ID NO: 27 - DNA, 621 bp, Echovirus
  CDS (1)..(621)
gtgttctggaccgaaggtaatgcaccagcacgaatgtccataccatttattagcatcgggaatgcttatagtaatttctatgatgggtggtcccattttacacaagatggtggctatgggtacaacacgcttaacaaaatgggcaaaatttatgtcaggcatgtgaacaaacaaacccccactgacgtgactagcactattagaatttactttaaacccaaacatgtgcgggcttggataccacgtccacctagactgtgtcaatacaagaataaagcaaacgtgaattttgaagttactagcgttacaactgctagaactaacctaaatgatgtcccgacacccagccacagtagcagtgtacatttgcgcctaagcactcaaggggcgtctgggcatcaagcaggtgctgcatacgttggcaattaccgggtggttaacagacacctagctacgcaacaggattggcaaaactgtgtatgggaagactacaacagagacctcttagtgagcactacgacagcacatggatgtgacaccattgctagatgccagtgtactacaggtgtgtacttctgtgcttcgaaaaacaagcattacccagtgagttttgaaggtcca

SEQ ID NO: 28 - protein, 207 aa, Echovirus
VFWTEGNAPARMSIPFISIGNAYSNFYDGWSHFTQDGGYGYNTLNKMGKIYVRHVNKQTPTDVTSTIRIYFKPKHVRAWIPRPPRLCQYKNKANVNFEVTSVTTARTNLNDVPTPSHSSSVHLRLSTQGASGHQAGAAYVGNYRVVNRHLATQQDWQNCVWEDYNRDLLVSTTTAHGCDTIARCQCTTGVYFCASKNKHYPVSFEGP

SEQ ID NO: 29 - DNA, 645 bp, Echovirus
  CDS (1)..(645)
tggattgagggaaacgccccccccagaatgtccatcccttttatgagtgtgggaaacgcatatagcaacttctacgatggatggtctcacttttcgcaaacaggtgtgtacggtttcaacactctcaacaacatggggaagttatatttcagacatgtgaacgacaagacaattagcccaatcacaagcaaagtccgcatctacttcaaaccaaaacacgtgaaagcttgggtaccccgcccacctagactgtgtgagtacacgcacaaagacaatgtggattttgaacccaaaggcgttaccacatcccgcacccaactaacaattagcaactccacacacatggaaaattatggtgcctttggacaacaatctggagccgtctacgtgggcaattatagactagtaaacagacacctagccacgcacaatgattggcagaactgtgtatgggaagactacaatagagaccttttagttagcaccaccacagcccatggatgtgacactattgctaggtgccaatgcaccactggtgtatacttttgtgcttccaaaaacaagcactatcccgtggcatttgagggacctggtctagtggaagtgcaggagagcgagtattaccctaaaagatatcagtctcat

SEQ ID NO: 30 - protein, 215 aa, Echovirus
WIEGNAPPRMSIPFMSVGNAYSNFYDGWSHFSQTGVYGFNTLNNMGKLYFRHVNDKTISPITSKVRIYFKPKHVKAWVPRPPRLCEYTHKDNVDFEPKGVTTSRTQLTISNSTHMENYGAFGQQSGAVYVGNYRLVNRHLATHNDWQNCVWEDYNRDLLVSTTTAHGCDTIARCQCTTGVYFCASKNKHYPVAFEGPGLVEVQESEYYPKRYQSH

SEQ ID NO: 31 - DNA, 750 bp, Coxsackievirus A
  CDS (1)..(750)
tggacagaaggaaatgcgccagcgcgaatgtcaatccccttcatcagcatagggaatgcttacagcaacttctatgacggatggtcaaacttcgaccagaagggctcatatgggtacaacacactaaacaacctaggtcacatatatgtgaggcatgtgagcggtagtagccctcatcccatcacgagtaccatcagaatatactttaaaccaaagcacactagagcctgggtgccgcggcctcctcgactgtgtcagtacaagaaagcattcagtgtggattttacaccaacgcccatcactgacactaggaaagacatcaacaccgtaacatcaatacaaagtgggcgtcgaggcgacatgtctgcactaaacacccacggagcgtttggacagcaatctggagcagtgtatgtgggaaattatagagtagtgaacagacatctcgccacacacaacgattggcaaaggtgtgtgtgggaaaactataatagggacctcctggtcagcaccaccacagctcacggatgtgacaccatcgctagatgtcaatgcactactggaatatactactgtgcctccaagaataaacactaccccgtagcttttgagggacccggtctggtggaggtgcaggagaatgaatactaccccaagagataccaatctcatgttcttctcgccgcggggttctctgagccaggggattgtggcggcattcttaggtgtgaacatggcgtcataggcatt

SEQ ID NO: 32 - protein, 250 aa, Coxsackievirus A
WTEGNAPARMSIPFISIGNAYSNFYDGWSNFDQKGSYGYNTLNNLGHIYVRHVSGSSPHPITSTIRIYFKPKHTRAWVPRPPRLCQYKKAFSVDFTPTPITDTRKDINTVTSIQSGRRGDMSALNTHGAFGQQSGAVYVGNYRVVNRHLATHNDWQRCVWENYNRDLLVSTTTAHGCDTIARCQCTTGIYYCASKNKHYPVAFEGPGLVEVQENEYYPKRYQSHVLLAAGFSEPGDCGGILRCEHGVIGI

SEQ ID NO: 33 - DNA, 732 bp, Echovirus
  CDS (1)..(732)
tggacagagggcaacgcccccccacgcatgtccattccattcataagtatagggaatgcgtacagtaatttttacgatggttggtcacacttttcacaaaacggggtgtatgggtacaacgcattaaacaatatgggcaagttgtacgcacgacacgtgaacaaagacacaccataccaaatgtctagcacgatccgtgtgtactttaagcctaagcacatcagagtctgggtgccgcgaccaccgcgcttgtgtccctacatcaagtccaacaacgttaattttgacccgaccaacctaactgactcaagatcaagcataacatacgtgcccgatactatccgtccagaggttcgcacaaccggtaggtttggccaacagtctggcgcaatctacgtgggaaattatagagtggtgaacaggcatcttgccactcacaatgactggcaaaattgtgtgtgggaagagtataacagagacctccttgtgagtaccaccacagcccatggatgtgacaccatcgccagatgccagtgtacgacaggtgtgtacttttgtgcctcaagaaacaaacattatccagtcacctttgaagggccaggcttagtagaagtccaggagagtgaatactaccccaagaggtatcagtcccatgtcctcttggcagctgggttctccgagccaggtgactgtggcggtatactgaggtgtgagcatggtgctatcggtctt

SEQ ID NO: 34 - protein, 244 aa, Echovirus
WTEGNAPPRMSIPFISIGNAYSNFYDGWSHFSQNGVYGYNALNNMGKLYARHVNKDTPYQMSSTIRVYFKPKHIRVWVPRPPRLCPYIKSNNVNFDPTNLTDSRSSITYVPDTIRPEVRTTGRFGQQSGAIYVGNYRVVNRHLATHNDWQNCVWEEYNRDLLVSTTTAHGCDTIARCQCTTGVYFCASRNKHYPVTFEGPGLVEVQESEYYPKRYQSHVLLAAGFSEPGDCGGILRCEHGAIGL

SEQ ID NO: 35 - DNA, 750 bp, Echovirus
  CDS (1)..(750)
tggactgaaggcaatgcacctcccaggatgtccattccctttatgagcatcggaaacgcgtatagcaatttctatgatgggtggtcccacttttctcagaatggggtttatggttacaacacattaaaccacatggggcagctgtacatgcgccacgtgaacgggccatcaccactgccaatgaccagcacggtgagagtgtacttcaaaccaaagcatgtgaaggcatgggtgccacggcctccaaggctatgtcagtataagaatgcatcgactgtgaacttctcatctacaaacattactgacaagagagacagcatcacctacgtaccagacaccgtcaagcctgacgtgtcgaaccatggcgcctttggacaacagtctggagctgtttacgtgggcaactacagagtagtcaacagacaccttgctacacacaacgattggcaaaactgtgtgtgggaagattataacagggacctgctggtgagcaccactacagcacacgggtgcgacactattgccagatgtcaatgcaatgctggggtgtacttctgtgcttcaaaaaataaacactacccgattgtcttcgaaggtccaggtcttgtggaggtgcaggagagtgaatattaccctaagagatatcaatcacatgtgttgctcgctgcaggcttttctgaaccaggagattgcggtggtatcctgagatgtgaacacggggtcattggcatcgtgaccatgggtggtgaa

SEQ ID NO: 36 - protein, 250 aa, Echovirus
WTEGNAPPRMSIPFMSIGNAYSNFYDGWSHFSQNGVYGYNTLNHMGQLYMRHVNGPSPLPMTSTVRVYFKPKHVKAWVPRPPRLCQYKNASTVNFSSTNITDKRDSITYVPDTVKPDVSNHGAFGQQSGAVYVGNYRVVNRHLATHNDWQNCVWEDYNRDLLVSTTTAHGCDTIARCQCNAGVYFCASKNKHYPIVFEGPGLVEVQESEYYPKRYQSHVLLAAGFSEPGDCGGILRCEHGVIGIVTMGGE

SEQ ID NO: 37 - DNA, 690 bp, Echovirus
  CDS (1)..(690)
tggactgaggggaatgctccacccaggatgtccatcccatttattagcatcggcaacgcatacagcagcttctatgatgggtggtcacactttgacagcaagggtgcatacggtttcaacactttgaacaaaatggggcacatctattgtagacacgtgaacaaggaaacgcctgctgaagttaccagttacatcaggatttactttaaacctaagcatgtcagagcatgggtgccaaggcctccgcggctatgccagtacaaaaacaaggcaaatgtgaactttgacgccacagcggttactgatacgcgtgctaccattaatactgttcctgtatcaagtcatggcggtgaacgccgtggtgacctggctgtattgaatacgcatggtgcttttggccaacaatccggagccatatatgtgggcaattacagggtggttaacagacatctagctacgcacacggactggcagaactgcgtttgggaagattataatagggatctcctagtgagtacaacaaccgctcatggctgtgacacgatagctagatgtcaatgtaaagcaggtgtatactactgccaatccaagagcaagcattatccggtgtcctttgaggggccaggtctggtggaagtacaggaaagtgagtactaccccaagagataccaatctcatgtgctccttgcc

SEQ ID NO: 38 - protein, 230 aa, Echovirus
WTEGNAPPRMSIPFISIGNAYSSFYDGWSHFDSKGAYGFNTLNKMGHIYCRHVNKETPAEVTSYIRIYFKPKHVRAWVPRPPRLCQYKNKANVNFDATAVTDTRATINTVPVSSHGGERRGDLAVLNTHGAFGQQSGAIYVGNYRVVNRHLATHTDWQNCVWEDYNRDLLVSTTTAHGCDTIARCQCKAGVYYCQSKSKHYPVSFEGPGLVEVQESEYYPKRYQSHVLLA

SEQ ID NO: 39 - DNA, 762 bp, Echovirus
  CDS (1)..(762)
tggacagagggcaacgctcccccacgcatgtccattccattcataagtatagggaatgcgtacagtaatttttacgatggttggtcacacttttcacaaaacggggtgtatgggtacaacgcattgaacaatatgggcaagttgtacgcacgacacgtgaacaaagacacaccataccaaatgtctagcacgatccgtgtgtactttaagcccaagcacattagagtctgggtgccgcgaccaccgcgcttgtgtccctatatcaagtccagcaacgttaattttgacccgaccaacctaactgactcaagatcaagcataacatacgtgcccgatactatccgtccagaggttcgcacaaccggtaggtttggccaacagtctggcgcaatctacgtgggaaattatagagtggtgaacagacatctcgccactcacaatgactggcaaaattgtgtgtgggaagagtataacagagacctccttgtgagtaccaccacagcccatggatgtgacaccatcgccagatgccagtgtacgacaggtgtgtacttttgtgcctcaagaaacaaacattatccagtcacctttgaagggccaggtttagtagaagtccaggagagtgaatactaccccaagaggtatcagtcccatgtcctcttagcagccgggttctccgagccaggtgattgtggcggtatattgaggtgtgagcatggtgttatcggtctcgttaccatgggtggcgaaggtgttgtggga

SEQ ID NO: 40 - protein, 254 aa, Echovirus
WTEGNAPPRMSIPFISIGNAYSNFYDGWSHFSQNGVYGYNALNNMGKLYARHVNKDTPYQMSSTIRVYFKPKHIRVWVPRPPRLCPYIKSSNVNFDPTNLTDSRSSITYVPDTIRPEVRTTGRFGQQSGAIYVGNYRVVNRHLATHNDWQNCVWEEYNRDLLVSTTTAHGCDTIARCQCTTGVYFCASRNKHYPVTFEGPGLVEVQESEYYPKRYQSHVLLAAGFSEPGDCGGILRCEHGVIGLVTMGGEGVVG

SEQ ID NO: 41 - DNA, 630 bp, Coxsackievirus B
  CDS (1)..(630)
acagagggcaacgcaccaccaagaatgtcgataccgttcatgagcataggtaatgcgtacaccatgttctatgacggctggtcaaatttctctagggatggcatatatggttacaattcactaaataacatgggaactatttatgcgcgtcatgtcaacgattccagcccgggtggtttaaccagtaccattcgcatctacttcaaacccaagcacgtcaaggcgtatgtgccgcgtccaccacgcttgtgccaatacaagaaggcaaaaaacgtgaactttgaagttgaggccgttaccaccgagcgtgcgagtttagtgaccaccggtccacacggacaacagtctggtgcagtgtatgtgggcaactacagaatcataaacagacatttggcaacaaactttgattggcaaaattgcgtgtgggaggattacaacagggacctcctagtgagcacaaccacagcgcatgggtgcgataccatcgctagatgccagtgtaccacgggcgtgtacttttgtgcatcgaagaacaagcactatccagttgcctttgagggcccaagtcttgtggaagttcaagaaagtgaatattaccccaaaaggtaccattcccatgtactccttgca

SEQ ID NO: 42 - protein, 210 aa, Coxsackievirus B
TEGNAPPRMSIPFMSIGNAYTMFYDGWSNFSRDGIYGYNSLNNMGTIYARHVNDSSPGGLTSTIRIYFKPKHVKAYVPRPPRLCQYKKAKNVNFEVEAVTTERASLVTTGPHGQQSGAVYVGNYRIINRHLATNFDWQNCVWEDYNRDLLVSTTTAHGCDTIARCQCTTGVYFCASKNKHYPVAFEGPSLVEVQESEYYPKRYHSHVLLA

SEQ ID NO: 43 - DNA, 519 bp, Echovirus
  CDS (1)..(519)
tggaccgagggcaatgcacctcccaggatgtctatcccctttatgagcatcgggaacgcttatagcaacttctatgatggatggtcccacttttctcagaatggggtttatggttacaatacattaaaccacatggggcagttgtacgtgcgccacgtgaacggaccatcgccactgccaatgaccagcacagtgagagtgtacttcaaaccaaaacatgtgaaggcgtgggtgccacgacccccaaggctatgtcagtacaagaatgcatcaaccgtgaacttctcatctacaaatattactgacaagagagacagcatcacctacataccagacactgtaaagcctgacgtatcgaactacggggcgtttggtcaacagtccggtgccgtgtacgtgggcaattatagagtggttaacagacatttagcaacgtacaatgattggcaaaactgtgtgtgggaagactacaacagagatctcttggtcagcactaccacagcacatgggtgcgacacc

SEQ ID NO: 44 - protein, 173 aa, Echovirus
WTEGNAPPRMSIPFMSIGNAYSNFYDGWSHFSQNGVYGYNTLNHMGQLYVRHVNGPSPLPMTSTVRVYFKPKHVKAWVPRPPRLCQYKNASTVNFSSTNITDKRDSITYIPDTVKPDVSNYGAFGQQSGAVYVGNYRVVNRHLATYNDWQNCVWEDYNRDLLVSTTTAHGCDT

SEQ ID NO: 45 - DNA, 744 bp, Echovirus
  CDS (1)..(744)
tggaccgagggcaatgcacctcccaggatgtctatcccctttatgagcatcgggaacgcttatagcaacttctatgatgggtggtcccacttttctcagaatggggtttatggttacaatacattaaaccacatggggcagttgtacgtgcgccacgtgaacgggccatcgccactgccaatgaccagcacagtgagagtgtacttcaaaccaaaacatgtgaaggcgtgggtgccacgacccccaaggctatgtcagtacaagaatgcatcaaccgtaaacttctcatctacaaatattactgacaagagaaatagcatcacctacataccagacactgtaaagcctgacgtgtcgaactacggggcgtttggtcaacagtccggtgccgtgtacgtgggcaattatagagtggttaacagacatttagcaacatacaatgattggcaaaactgtgtgtgggaagactacaacagagatctcttggtcagcactaccacagcacatggatgcgacaccattgctaggtgtcagtgcatggcaggggtgtacttctgtcgatccaggaacaagcattacccagtttgttttgagggtccaggcctagtggaggttcaagagagtgagtactaccccaagagatatcagtcccatgtgttgttagcagcaggactttcagaacccggagactgtggtggcatactaaggtgccagcatggcgtcgttggcatagtgaccatggga

SEQ ID NO: 46 - protein, 248 aa, Echovirus
WTEGNAPPRMSIPFMSIGNAYSNFYDGWSHFSQNGVYGYNTLNHMGQLYVRHVNGPSPLPMTSTVRVYFKPKHVKAWVPRPPRLCQYKNASTVNFSSTNITDKRNSITYIPDTVKPDVSNYGAFGQQSGAVYVGNYRVVNRHLATYNDWQNCVWEDYNRDLLVSTTTAHGCDTIARCQCMAGVYFCRSRNKHYPVCFEGPGLVEVQESEYYPKRYQSHVLLAAGLSEPGDCGGILRCQHGVVGIVTMG

SEQ ID NO: 47 - DNA, 726 bp, Echovirus
  CDS (1)..(726)
ttctggaccgagggtaatgcacctcccaggatgtctatcccctttatgagcattgggaacgcttatagcaacttttatgacgggtggtcccacttttctcagaatggggtatatggttacaatacattaaaccacatggggcagttgtacatgcgccacgtgaatgggccatcgccactgccaatgaccagcacagtgagagtgtacttcaaaccaaagcatgtgaaggcatgggtgccacgacccccaaggctatgtcagtacaagaatgcatcaaccgtgaacttttcatctacaaatattactgacaagagaaacagcatcacctacataccagacactgtcaaacctgacgtgtcgaactacggggcgtttggtcagcagtcaggcgctgtgtacgtgggcaattatagagtggttaatagacatttggcaacatataatgattggcaaaactgtgtgtgggaagactataacagagatctcttggtcagcactaccacagcacatgggtgcgacaccattgctaggtgtcagtgcatggcaggggtgtatttttgtcgatccaggaacaagcattacccagttagctttgagggtccaggcctggtggaggttcaagagagtgagtactaccccaagagatatcagtcccatgtgttgttagcggcaggactttcagaacccggagactgtggcggcatactaagatgtgaacatggcgtc

SEQ ID NO: 48 - protein, 242 aa, Echovirus
FWTEGNAPPRMSIPFMSIGNAYSNFYDGWSHFSQNGVYGYNTLNHMGQLYMRHVNGPSPLPMTSTVRVYFKPKHVKAWVPRPPRLCQYKNASTVNFSSTNITDKRNSITYIPDTVKPDVSNYGAFGQQSGAVYVGNYRVVNRHLATYNDWQNCVWEDYNRDLLVSTTTAHGCDTIARCQCMAGVYFCRSRNKHYPVSFEGPGLVEVQESEYYPKRYQSHVLLAAGLSEPGDCGGILRCEHGV

SEQ ID NO: 49 - DNA, 717 bp, Echovirus
  CDS (1)..(717)
tggaccgagggtaatgcacctcccaggatgtctatcccctttatgagcattgggaacgcttatagcaacttttatgacgggtggtcccacttttctcagaatggggtttatggttacaatacattaaaccacatggggcagttgtacatgcgccacgtgaacgggccatcgccactgccaatgaccagcacagtgagggtgtatttcaaaccaaagcatgtgaaggcatgggtgccacgacccccaaggctatgtcaatacaagaatgcatcaaccgtgaacttttcatctacaaatattactgacaagagaggcagcatcacctacataccagacaccgtcaaacctgacgtgtcgaactacggggcgtttggtcagcagtcaggcgctgtgtacgtgggcaattatagagtggttaacagacatttggcaacatataatgattggcaaaattgtgtgtgggaagactataacagagatctcttggtcagcactaccacagcacatgggtgtgacaccattgctaggtgtcggtgcatggcaggggtgtatttttgtcgatccaggaacaagcattacccagttagctttgagggtccaggcctggtggaggttcaagagagtgagtactaccccaagagatatcagtcccatgtgttgttagcggcaggactttcagaacccggagactgtggtggcatactaagatgtgaacat

SEQ ID NO: 50 - protein, 239 aa, Echovirus
WTEGNAPPRMSIPFMSIGNAYSNFYDGWSHFSQNGVYGYNTLNHMGQLYMRHVNGPSPLPMTSTVRVYFKPKHVKAWVPRPPRLCQYKNASTVNFSSTNITDKRGSITYIPDTVKPDVSNYGAFGQQSGAVYVGNYRVVNRHLATYNDWQNCVWEDYNRDLLVSTTTAHGCDTIARCRCMAGVYFCRSRNKHYPVSFEGPGLVEVQESEYYPKRYQSHVLLAAGLSEPGDCGGILRCEH

SEQ ID NO: 51 - DNA, 609 bp, Coxsackievirus B
  CDS (1)..(609)
acccaccaaataatgtatgtgccaccaggtggcccagtgccagataaggttgactcatatgtgtggcagacgtccacaaatcccagtgttttctggaccgaaggcaatgcaccgccacgcatgtccattccatttttgagcattggcaacgcatactctaacttttatgatggctggtcagagtttgccagaagcggagtgtatggcataaacactttgaacaatatggggaccctttacgcaagacatgtgaacgttgggagcacaggaccaataaaaagtactattagaatctacttcaagccaaagcacgttaaggcatggatacctagacctccaaggctgtgtcagtatgaaaaagcaaagaatgtaaattttcaacccagtggtgtaacaacaaccagacagagcatcacagcaatgacaaatactggggcgttcgggcagcaatcgggggcaatatacgtgggcaactatagggtggtgaatagacatctggctacgttcacagattggcagaattgtgtgtgggaagattataacagggatctacttggtagcactactacgggccatgggtgtgatgtcattgccagatgccggtgtaca

SEQ ID NO: 52 - protein, 203 aa, Coxsackievirus B
THQIMYVPPGGPVPDKVDSYVWQTSTNPSVFWTEGNAPPRMSIPFLSIGNAYSNFYDGWSEFARSGVYGINTLNNMGTLYARHVNVGSTGPIKSTIRIYFKPKHVKAWIPRPPRLCQYEKAKNVNFQPSGVTTTRQSITAMTNTGAFGQQSGAIYVGNYRVVNRHLATFTDWQNCVWEDYNRDLLGSTTTGHGCDVIARCRCT

SEQ ID NO: 53 - DNA, 720 bp, Echovirus
  CDS (1)..(720)
aatgcccctcccaggatgtcgataccattcatgagtgttggtaacgcatattgcaacttttatgatggatggtctcatttcagccagagcggcgtgtacggatacactactttgaacaacatgggacacttgtattttagacatgtgaacaaatcgactgcatatccagttaacagtgttgcccgcgtctattttaagcccaagcacgtgaaggcatgggtgcctcgtgcaccacgcttgtgcccgtatctgtatgcaagaaatgtcaatttttatgtgcaaggggtaaccgaatctcggggcaaaattactcttgaccgatcaactcacaatcctttgttaaacactggtgtttttgggcaacaatcaggggctgcatatgtgggcaactacagagtagtcaacagacacctcgccacacacgttgactggcagaattgcgtgtgggaagactacaatagagacttgttagttagcaccaccacagcccacgggtgtgacaccatagctaggtgccagtgctccacaggtgtgtacttttgcgcatccaggaataaacattacccagtgtcatttgaagggcccgggctcgttgaggtgcaggagagtgagtattacccaaaaaggtaccagtcacatgtgttgctcgccgcaaggttttctgagccaggtgactgtgggggcatcttgagatgcgagcatggcgtcatagggcttgtg

SEQ ID NO: 54 - protein, 240 aa, Echovirus
NAPPRMSIPFMSVGNAYCNFYDGWSHFSQSGVYGYTTLNNMGHLYFRHVNKSTAYPVNSVARVYFKPKHVKAWVPRAPRLCPYLYARNVNFYVQGVTESRGKITLDRSTHNPLLNTGVFGQQSGAAYVGNYRVVNRHLATHVDWQNCVWEDYNRDLLVSTTTAHGCDTIARCQCSTGVYFCASRNKHYPVSFEGPGLVEVQESEYYPKRYQSHVLLAARFSEPGDCGGILRCEHGVIGLV

SEQ ID NO: 55 - DNA, 831 bp, Coxsackievirus A
  CDS (1)..(831)
tggacagaaggaaacgcaccagcgcgcatgtcaatcccctttatcagcatagggaatgcttatagcaacttttatgacgggtggtcgaacttcgatcagaagggctcatacgggtacaatacattaaacaacctaggccacatatacgtgaggcatgtgagcgggagtagccctcatccaatcacgagtaccatcaggatatattttaaaccaaagcatactagagcctgggtgccacgacctccgcgattatgtcagtataagaaggcattcagtgtagattttacaccaactcctattactgacaccaggaaagacatcaacaccgtaacctcaatacaaagtgggcgccgaggtgacatgtccgcactaaacacccatggggcgtttggacagcaatctggagcggtgtatgtgggaaactatagagtggtgaacagacaccttgccacacgcaatgactggcaaaactgtgtgtgggaggattataatagagaccttctagtgagcacaaccaatgcacatggctgtgacactatagccagatgtcaatgtaccacaggtgtgtatttctgtgcttcaaggaataagcattacccagtgagttttgaaggaccaggcttagtggaagttcaggaaagtgagtactaccccaagaggtaccaatcccatgttctcctggcagcgggtttctctgagcccggtgattgtggtggcatcttgaggtgtgagcacggagtcatcggcattgtgactatgggtggtgaaggcgtcgtcggttttgctgatgtgcgagacctcttgtggttagaagatgatgctatggaacag

SEQ ID NO: 56 - protein, 277 aa, Coxsackievirus A
WTEGNAPARMSIPFISIGNAYSNFYDGWSNFDQKGSYGYNTLNNLGHIYVRHVSGSSPHPITSTIRIYFKPKHTRAWVPRPPRLCQYKKAFSVDFTPTPITDTRKDINTVTSIQSGRRGDMSALNTHGAFGQQSGAVYVGNYRVVNRHLATRNDWQNCVWEDYNRDLLVSTTNAHGCDTIARCQCTTGVYFCASRNKHYPVSFEGPGLVEVQESEYYPKRYQSHVLLAAGFSEPGDCGGILRCEHGVIGIVTMGGEGVVGFADVRDLLWLEDDAMEQ

SEQ ID NO: 57 - DNA, 666 bp, Echovirus
  CDS (1)..(666)
acagagggcaacgcccctgctcgcatgtcaatacccttcatgagtgttggcaacgcttactgcaacttctatgatggatggtctcacttttcacaatctggtgtttatggcttcaccaccctcaacaacatgggccaattgttcttccgccatgtgaataaagacacccttggtccctataacagtactgtgagagtttacttcaaaccaaagcacattaaagcatgggtgcccaggccaccacgcctgtgtgactacgtatacgcacacaacgttgacttcaaccctaaaggagtcacggagagtagagacaagatcacactggagagagatgaccacttgccctcaatggttagccatggggcatttggacagcaatctggtgctgtgtacgtaggcaattacagggttgtcaatagacacctagcaactcacgctgactggcaaaactgcgtgtgggaggactacaatagagatttgctggttagcaccaccacagcccatggttgcgacactatagctagatgtcagtgcaccacaggtgtctatttttgcgcctcgagaaacaagcattatccagtaagttttgaggggcccggtttagttgaggtgcaggagagtgagtactaccctaaaagataccagtcccatgtattgctagcagca

SEQ ID NO: 58 - protein, 222 aa, Echovirus
TEGNAPARMSIPFMSVGNAYCNFYDGWSHFSQSGVYGFTTLNNMGQLFFRHVNKDTLGPYNSTVRVYFKPKHIKAWVPRPPRLCDYVYAHNVDFNPKGVTESRDKITLERDDHLPSMVSHGAFGQQSGAVYVGNYRVVNRHLATHADWQNCVWEDYNRDLLVSTTTAHGCDTIARCQCTTGVYFCASRNKHYPVSFEGPGLVEVQESEYYPKRYQSHVLLAA

SEQ ID NO: 59 - DNA, 774 bp, Coxsackievirus A
  CDS (1)..(774)
gtgtcagttcccttcatgtcaccagccagtgcatatcaatggttttacgatggctaccctacttttggagagcacctccaagcaaatgacctagattatggccagtgtcctaataacatgatgggcacctttagcattaggacagtagggactgaaaagtcaccacactccattaccctgagagtgtacatgagaattaaacacgtcagggcgtggataccaaggcctctgagaaaccaaccctatctatttaagaccaacccaaattataaaggaaatgatatcaaatgcactagcaccagtagagacaagatcacgaccttagggaagtttggacagcaatcaggcgccatatatgtgggcaattatagggtagtgaatcgacacctcgcaacacacaacgattgggcaaacctcgtgtgggaagacagctctagggatctgttagtctcctccaccaccgcccaggggtgcgatactattgctagatgcaattgccagaccggggtgtactactgtaactccaagagaaaacattatccagttagtttcactaagcctagcttgatatttgtagaagctagcgagtactacccagctaggtaccagtcacacctcatgcttgctcagggtcactcagaacctggcgactgtggtggcattcttagatgccagcacggtgtagtaggacttgtttccactggtgggaatggccttgtagggtttgctgatattagagaccttttatggctagatgaggaagcaatggaacag

SEQ ID NO: 60 - protein, 258 aa, Coxsackievirus A
VSVPFMSPASAYQWFYDGYPTFGEHLQANDLDYGQCPNNMMGTFSIRTVGTEKSPHSITLRVYMRIKHVRAWIPRPLRNQPYLFKTNPNYKGNDIKCTSTSRDKITTLGKFGQQSGAIYVGNYRVVNRHLATHNDWANLVWEDSSRDLLVSSTTAQGCDTIARCNCQTGVYYCNSKRKHYPVSFTKPSLIFVEASEYYPARYQSHLMLAQGHSEPGDCGGILRCQHGVVGLVSTGGNGLVGFADIRDLLWLDEEAMEQ

SEQ ID NO: 61 - DNA, 717 bp, Coxsackievirus A
  CDS (1)..(717)
gcatatcaaaggttttacgatggttaccctacttttggagagcaccttcaagcaaatgacctatattatggccagtgccctaataacatgatgggcacctttagcattaggacagtagggactgaaaagtcaccacactccattaccctgagagtgtacatgagaattaaacacgtcagggcgtggataccaaggcctctgagaaaccaaccctatctatttaagaccaacccaaattataaaggaaatgatatcaaatgcactagcaccagtagagacaagatcacgaccttagggaagtttggacagcaatcaggcgccatatatgtgggcaattatagggtagtgaatcgacacctcgcaacacacaacgattgggcaaacctcgtgtgggaagacagctctagggatctgttagtctcctccaccaccgcccaggggtgcgatactattgctagatgcaattgccagaccggggtgtactactgtaactccaagagaaaacattatccagttagtttcactaagcctagcttgatatttgtagaagctagcgagtactacccagctaggtaccagtcacacctcatgcttgctcagggtcactcagaacctggcgactgtggtggcattcttagatgccagcacggtgtagtaggacttggttccactggtgggaatggccttgtagggtttgctgatattagagaccttttt

SEQ ID NO: 62 - protein, 239 aa, Coxsackievirus A
AYQRFYDGYPTFGEHLQANDLYYGQCPNNMMGTFSIRTVGTEKSPHSITLRVYMRIKHVRAWIPRPLRNQPYLFKTNPNYKGNDIKCTSTSRDKITTLGKFGQQSGAIYVGNYRVVNRHLATHNDWANLVWEDSSRDLLVSSTTAQGCDTIARCNCQTGVYYCNSKRKHYPVSFTKPSLIFVEASEYYPARYQSHLMLAQGHSEPGDCGGILRCQHGVVGLGSTGGNGLVGFADIRDLF

SEQ ID NO: 63 - DNA, 756 bp, Unknown
  one of six Madagascan enterovirus isolates, not neutralizable by PDVM pools, therefore untypeable
  CDS (1)..(756)
tacacttatggcacagcaccacctaggatttcaataccttacatcggtatcgccaatgcttactcccacttctacgatgggtatgcagttgtgccgttaaaagattccgtacaagactcaggagctgcgtattacggtgcagtgtccattaatgattttggccttcttgcagttcgagtggttaatgagcataaccctgtcagagtatcatcgaaggttagagtatacatgaaaccaaaacatgtcagagtgtggtgtccacgcccaccacgtgccgtggagtactacggacctggggtggattacaaggcagacacattaacaccactaccaaccaagaatctaaccacatatggctttggtcaccaaaacaaggcaatatatgttgcagggtataagatagtgaattaccacttggccacggcagaggattttaggcactgtgttagaagcatgtgggagcgtgacatcatgatagtggagtccaaagcacaagggaaagaccaaattgccagatgtgcgtgccgatcaggagtgtactactgtgaatcaagaggaaagcactaccccattacaattacacaacccacctttcagtggatggagaagaatgatttctacccagcacgctatcaatcacacatgacaattggccacggttttgcagcccctggggactgtggtggcatcttacggtgccaacacggtgtcatgggcctaataactgctggtggtaatggactggtagcattcgca

SEQ ID NO: 64 - protein, 252 aa, Unknown
  one of six Madagascan enterovirus isolates, not neutralizable by PDVM pools, therefore untypeable
YTYGTAPPRISIPYIGIANAYSHFYDGYAVVPLKDSVQDSGAAYYGAVSINDFGLLAVRVVNEHNPVRVSSKVRVYMKPKHVRVWCPRPPRAVEYYGPGVDYKADTLTPLPTKNLTTYGFGHQNKAIYVAGYKIVNYHLATAEDFRHCVRSMWERDIMIVESKAQGKDQIARCACRSGVYYCESRGKHYPITITQPTFQWMEKNDFYPARYQSHMTIGHGFAAPGDCGGILRCQHGVMGLITAGGNGLVAFA

SEQ ID NO: 65 - DNA, 726 bp, Unknown
  one of six Madagascan enterovirus isolates, not neutralizable by PDVM pools, therefore untypeable
  CDS (1)..(726)
tacacttatcgcacagcaccacctaggatttcaataccttacatcggtatcgccaatgcttactcccacttctacgatgggtatgcagttgtgccgctaaaagattccgcacaagactcaggagctgcgtattacggtgcagtgtccattaatgactttggccttcttgcagttcgagtggttaacgagcataaccctgtcagagtatcatcgaaggttagagtatacatgaaaccaaaacacgtcagagtgtggtgtccacgcccaccacgtgccgtggagtactacggacctggggtggattacaaggcagacacattaacaccactaccaactaagaatctaaccacatatggctttggtcaccaaaacaaggcaatatatgttgcagggtataagatagtgaattaccacctggccacggcagaggattttaggcactgtgttagaagcatgtgggagcgtgacatcatgatagtggagtccaaagcacaagggagagaccaaattgccagatgtgcgtgccgatcaggagtgtactactgtgaatcaagaggaaagcactaccccattacaattacacaacccacctttcagtggatggagaagaatgatttctacccagcacgctatcaatcacacatgacaattggccacggttttgcagcccctggggactgtggtggcatcttacggtgccaacacggtgtcatgggcctaataact

SEQ ID NO: 66 - protein, 242 aa, Unknown
  one of six Madagascan enterovirus isolates, not neutralizable by PDVM pools, therefore untypeable
YTYRTAPPRISIPYIGIANAYSHFYDGYAVVPLKDSAQDSGAAYYGAVSINDFGLLAVRVVNEHNPVRVSSKVRVYMKPKHVRVWCPRPPRAVEYYGPGVDYKADTLTPLPTKNLTTYGFGHQNKAIYVAGYKIVNYHLATAEDFRHCVRSMWERDIMIVESKAQGRDQIARCACRSGVYYCESRGKHYPITITQPTFQWMEKNDFYPARYQSHMTIGHGFAAPGDCGGILRCQHGVMGLIT

SEQ ID NO: 67 - DNA, 747 bp, Unknown
  one of six Madagascan enterovirus isolates, not neutralizable by PDVM pools, therefore untypeable
  CDS (1)..(747)
tacacttatggtacagcaccacctaggatttcaataccttacatcggtatcgccaatgcttactcccacttctacgatgggtatgcagttgtgccactgaaagattccacactagactcaggagctgcgtattatggtgcagtgtccattaacgactttggccttcttgcagttcgagtggtcaatgagcacaaccctgtcagagtatcatcaaaggttagagtatacatgaaaccaaaacatgtcagagtgtggtgtccacgcccaccacgtgccgtggagtattacggacctggagtggattacaaggcaaacacattaacaccactaccaaccaagaatctaaccacatatggttttggtcaccaaaacaaggcaatatatgttgcagggtataagatagtgaattaccacttggccactgcagaggattttaggcactgtgttagaagtatgtgggagcgtgacatcatggtggtggaatccaaagcacaggggaccgaccaaattgcccgatgtgcgtgtcgagcaggtgtatatttctgtgaatcaagaggaaaacactatcccataacaattacacagcctaccttccagtggatggagaaaaatgatttctacccagcacgctaccagtcacacatgactatcggttgtggttacgcagcccctggagactgtggtggtattttgcgatgtcaacacggtgtcatgggcctgataaccgccggtggtgatgggctggtg

SEQ ID NO: 68 - protein, 249 aa, Unknown
  one of six Madagascan enterovirus isolates, not neutralizable by PDVM pools, therefore untypeable
YTYGTAPPRISIPYIGIANAYSHFYDGYAVVPLKDSTLDSGAAYYGAVSINDFGLLAVRVVNEHNPVRVSSKVRVYMKPKHVRVWCPRPPRAVEYYGPGVDYKANTLTPLPTKNLTTYGFGHQNKAIYVAGYKIVNYHLATAEDFRHCVRSMWERDIMVVESKAQGTDQIARCACRAGVYFCESRGKHYPITITQPTFQWMEKNDFYPARYQSHMTIGCGYAAPGDCGGILRCQHGVMGLITAGGDGLV

SEQ ID NO: 69 - DNA, 813 bp, Unknown
  one of six Madagascan enterovirus isolates, not neutralizable by PDVM pools, therefore untypeable
  CDS (1)..(813)
gtattctacacttacgggactgctcccgcgagaatttccataccgtacgtggggatcgctaacgcatattctcacttttatgatggatttgctactgttcctattgaaggagtgaccacagacccaggtgcagcatattacggggcaacttccatcaatgatttcgggattctagcggtgagggtggtgaatgaccacaacccagttcaggtgtcttcaaagatcagagtttacatgaaaccaaagcatgtgcgcgtgtggtgcccaaggccaccaagagctgtcccatacttcggtccgggtgtggactacaagggagatgccttgacccccttatcaacaaagtctctaaccacttatggatttggacatcaaaataaagcagtgtacacggcgggctacaagatttgtaactaccaccttgcaacccaggaggatctgtcaaatgcagtgaatatcatgtggaacagagatcttttagtgacagaatccaaagcccagggcacggattccatagctagatgtacttgcaatgctggagtgtactactgtgagtctagacgaaagtattaccccgtttccttcattggtccaaccttccaatacatggaagcaaatgattactacccagcccggtatcaatctcacatgctaatcggctatggctttgcctcgcctggagactgtggcggtatcctcagatgtcaacatggagttattggaatcatcacagctggaggggaaggcctagtggccttttcagacatcagagatctttatgcatatgaagaggaagccatggagcaaggcatc

SEQ ID NO: 70 - protein, 271 aa, Unknown
  one of six Madagascan enterovirus isolates, not neutralizable by PDVM pools, therefore untypeable
VFYTYGTAPARISIPYVGIANAYSHFYDGFATVPIEGVTTDPGAAYYGATSINDFGILAVRVVNDHNPVQVSSKIRVYMKPKHVRVWCPRPPRAVPYFGPGVDYKGDALTPLSTKSLTTYGFGHQNKAVYTAGYKICNYHLATQEDLSNAVNIMWNRDLLVTESKAQGTDSIARCTCNAGVYYCESRRKYYPVSFIGPTFQYMEANDYYPARYQSHMLIGYGFASPGDCGGILRCQHGVIGIITAGGEGLVAFSDIRDLYAYEEEAMEQGI

SEQ ID NO: 71 - DNA, 786 bp, ORGANI SM: Coxsackievirus B
  CDS (1)..(786)
tggaccgaaggtaacgcaccacctcgaatgtctgtacccttcatgagtatcggaaacgcctatagcaccttttatgacggttggtcagatttttcatctaagggagtgtatggtttgaacaccctgaataatatggggtcattgtacattcggcacgtcaatgggcccaacccagtgcctatcactagtaccatacgtatttacctcaagccaaaacatgttaaagcatgggtgcctcgaccaccaagattgtgtcaatatgaaacatcaaggcaagtcaacttcaatgtgacaggcgtgactgagtccagagacaacataactaccatgaaaaccaccggagccttcggtcaacagtctggggcagcgtatgttggcaactatagggtggttaatagacacttggcgacccacaccgacgggcaaaattgcatttgggaggactacaacagggaccttcttgtgagcacaaccacggcgcatggatgcgacactatagcagggtgccagtgcaccaccggcgtgtacttctgtgcttcaaagaacaaacattaccccgtgagtttcgaaggaccagggctggtggaggttcagaagagcgaatattaccccaagagatatcaatcccacgtgctcctagctgctgggttctcagaaccaggtgactgtggtggtattttaagatgcgagcacggtgtcataggtttggtgaccatgggaggaaagggcgttgttggctttgctgatgttcgggacttaatatggctggaggatgatgctatggaa

SEQ ID NO: 72 - protein, 262 aa, Coxsackievirus B
WTEGNAPPRMSVPFMSIGNAYSTFYDGWSDFSSKGVYGLNTLNNMGSLYIRHVNGPNPVPITSTIRIYLKPKHVKAWVPRPPRLCQYETSRQVNFNVTGVTESRDNITTMKTTGAFGQQSGAAYVGNYRVVNRHLATHTDGQNCIWEDYNRDLLVSTTTAHGCDTIAGCQCTTGVYFCASKNKHYPVSFEGPGLVEVQKSEYYPKRYQSHVLLAAGFSEPGDCGGILRCEHGVIGLVTMGGKGVVGFADVRDLIWLEDDAME

SEQ ID NO: 73 - DNA, 699 bp, Unknown
  one of six Madagascan enterovirus isolates, not neutralizable by PDVM pools, therefore untypeable
  CDS (1)..(699)
ataccgtacgtggggattgctaacgcatattctaacttttatgatggatgtgctactgttcccattgaaggagagaccacagacccatgtgcagcatattacggggcaacttccatcaatgacttcgggattctagcggtgagggtggtgaatgaacacaacccagttcaggtgtcttcaaagatcagagtttacatgaaaccaaagcatgtgcgcgtgtggtgcccaaggccaccaagagctgtcccatactttggcccgggagtggactacaagggagatgccttgacccccttatcaacaaagtctctaaccacttatggatttggacatcaaaacaaagcagtgtacacggcaggctacaagatttgcaactaccaccttgcaacccaggaagatctgtcaaatgcagtgaatgtcatgtggaacagagatcttttggtgacagaatccaaagcccagggcacagattccatagctagatgtacttgcaatgctggagtgtactactgtgagtctagacgaaagtattaccccgtttccttcattggtccaacctttcaatacatggaagcaaatgattactatccagcccggtatcaatcccacatgctaatcggctatggctttgcctcgcctggagactgtggcggtatcctcagatgtcaacatggagttattggaatcatcacagctgga

SEQ ID NO: 74 - protein, 233 aa, Unknown
  one of six Madagascan enterovirus isolates, not neutralizable by PDVM pools, therefore untypeable
IPYVGIANAYSNFYDGCATVPIEGETTDPCAAYYGATSINDFGILAVRVVNEHNPVQVSSKIRVYMKPKHVRVWCPRPPRAVPYFGPGVDYKGDALTPLSTKSLTTYGFGHQNKAVYTAGYKICNYHLATQEDLSNAVNVMWNRDLLVTESKAQGTDSIARCTCNAGVYYCESRRKYYPVSFIGPTFQYMEANDYYPARYQSHMLIGYGFASPGDCGGILRCQHGVIGIITAG

SEQ ID NO: 75 - DNA, 783 bp, Unknown
  one of six Madagascan enterovirus isolates, not neutralizable by PDVM pools, therefore untypeable
  CDS (1)..(783)
ttctacacttatggcacagcaccacctagaatttcaataccttacatcggtatcgccaatgcttactctcacttctacgacgggtatgcagttgtgccgctgaaagattccacacaagactcaggagctgcgtattatggtgcggtgtccatcaatgactttggccttcttgcagttcgagtggttaatgaacacaaccctgtcagagtatcatcgaagattagagtatacatgaaaccaaaacatgttagggtgtggtgtccacgcccaccacgtgccgtggagtactacggacctggggtggattacaaggcaaacacattaacaccactaccaaccaagaatctaaccacatatggctttggtcaccaaaacaaggcaatatatgtggcagggtataagatagtgaattaccacttggccacggcagaagattttaggcactgtgttagaagcatgtgggagcgtgacatcatggtggttgaatccaaagcgcatgggactgaccaaattgctcgatgtgcgtgtcggactggtgtgtatttttgtgaatcaagaggaaaacattatcccataacaattacacaacctactttccagtggatggagaaaaatgatttctacccagcacgctaccaatcgcacatgactatcggttgtggttacgcagcccctggggactgtggtggtatcctacggtgtcaacacggtgtcatgggcctgataaccgccggtggcgatggactggtggcatttgcagacgtcagggatctctacgttgaa

SEQ ID NO: 76 - protein, 261 aa, Unknown
  one of six Madagascan enterovirus isolates, not neutralizable by PDVM pools, therefore untypeable
FYTYGTAPPRISIPYIGIANAYSHFYDGYAVVPLKDSTQDSGAAYYGAVSINDFGLLAVRVVNEHNPVRVSSKIRVYMKPKHVRVWCPRPPRAVEYYGPGVDYKANTLTPLPTKNLTTYGFGHQNKAIYVAGYKIVNYHLATAEDFRHCVRSMWERDIMVVESKAHGTDQIARCACRTGVYFCESRGKHYPITITQPTFQWMEKNDFYPARYQSHMTIGCGYAAPGDCGGILRCQHGVMGLITAGGDGLVAFADVRDLYVE

SEQ ID NO: 77 - DNA, 705 bp, Coxsackievirus B
  CDS (1)..(705)
acggaagggaatgctccaccaaggatgtcaattccatttatcagcattggcaatgcgtacagctgtttttatgatggttggacgcagttttcaagaaatggagtctatgggattaacactttaaataacatgggtactttgtacatgaggcacgttaacgaggcaggacagggcccaattaagagcaccgtcagaatatacttcaaacccaaacacgtgaaggcgtgggtgcctcgaccgccaaggctgtgtcagtacgagaaacaaaagaatgtcaacttcaaccccacaggagttaccacaagcagactaaacatcacaacaacaggtgcctttggtcagcaatcgggtgccgtatacgttggtaactacaggattgtgaacaggcaccttgctacacacaatgactggcaaaattgtgtgtgggaggattataatagagaccttctcgtgagcaccactacggcgcacgggtgtgacaccattgccaggtgccgatgtacgtcaggtgtgtacttttgtgcctccaagaacagacactaccctgtaatctttgagggacctggccttgctgaagtccaagaaagtgaatactaccctaaacggtaccaatcacatgtgcttcttgcagcaggcttctctgagcctggagattgtggaggcatcttgaggtgtgaacatggcgttattggtattgtgaccatg

SEQ ID NO: 78 - protein, 235 aa, Coxsackievirus B
TEGNAPPRMSIPFISIGNAYSCFYDGWTQFSRNGVYGINTLNNMGTLYMRHVNEAGQGPIKSTVRIYFKPKHVKAWVPRPPRLCQYEKQKNVNFNPTGVTTSRLNITTTGAFGQQSGAVYVGNYRIVNRHLATHNDWQNCVWEDYNRDLLVSTTTAHGCDTIARCRCTSGVYFCASKNRHYPVIFEGPGLAEVQESEYYPKRYQSHVLLAAGFSEPGDCGGILRCEHGVIGIVTM

SEQ ID NO: 79 - DNA, 777 bp, Coxsackievirus B
  CDS (1)..(777)
gtcttttggaccgaggggaatgcaccaccaagaatgtcaatcccattcattagcattggcaatgcatacagctgtttttacgatgggtggacgcaattttcgagaaatggggtctacggaatcaataccctgaacaacatgggcactctgtacatgcggcatgtcaatgaagcaggacagggaccaatcaagagtacagtcagaatatatttcaagcccaagcacgtaaaagcgtggataccacgaccaccaaggttgtgccagtatgagaagcaaaagaatgtcaactttaaccccacaggagtcaccgagagcaggctgaatattacgacaaccggggtttttgggcagcaatcaggggctgtgtacgtggggaactacaaagtggtaaacagacatttggcgactcaccatgattggcaaaactgcgtgtgggaagattacaacagggatctacttgttagcacaaccacagcccatgggtgcgacacaatagccagatgccattgtacatctggagtgtatttctgcaacagtagaaataagcactatccggtttcgtttgaaggcccggggttagttgaggttcaggaaagtgaatattacccaaaaagataccaatcccacgtgctccttgcaaccgggttttccaagccaggcgattgcggtggcatcttaaggtgcgagcatggtgtcattgggctagtgactatgggaggagagggtgtcgttggctttgctgacgtgcgcgaacttctgtggttagaggatgatgca

SEQ ID NO: 80 - protein, 259 aa, Coxsackievirus B
VFWTEGNAPPRMSIPFISIGNAYSCFYDGWTQFSRNGVYGINTLNNMGTLYMRHVNEAGQGPIKSTVRIYFKPKHVKAWIPRPPRLCQYEKQKNVNFNPTGVTESRLNITTTGVFGQQSGAVYVGNYKVVNRHLATHHDWQNCVWEDYNRDLLVSTTTAHGCDTIARCHCTSGVYFCNSRNKHYPVSFEGPGLVEVQESEYYPKRYQSHVLLATGFSKPGDCGGILRCEHGVIGLVTMGGEGVVGFADVRELLWLEDDA

SEQ ID NO: 81 - DNA, 651 bp, Echovirus
  CDS (1)..(651)
tggacagagggcaacgcccccccgcgtatgtccatcccatttataagtatagggaatgcgtacagtaatttttatgacggatggtcacacttttcacaaaacggggtatatgggtacaacgcattaaacaacatgggcaagttgtacgcacgacacgtgaataaagatacaccataccaaatgtctagcacgatccgcgtgtactttaagcccaagcacatcagagtctgggtgccgcgaccaccgcgcttgtgtccttatattaagtctagtaatgttaattttgaaccgaccaacctaactgactcaagatcaagcataacatacgtgcccgatactatccgtccagaagttcgcacagccggcaagtttggtcaacagtctggcgcaatctacgtgggaaattatagagtggtgaacagacatcttgccactcacagcgactggcaaaattgtgtgtgggaagaatataacagagacctcctcgtgagtaccaccacagcccatggatgtgacaccattgccagatgccagtgcacgacaggtgtgtacttttgtgcctcgagaaacaaacattatccagtcacctttgaaggaccaggtttggtagaagtccaggagagtgaatactaccccaagagatatcagtcc

SEQ ID NO: 82 - protein, 217 aa, Echovirus
WTEGNAPPRMSIPFISIGNAYSNFYDGWSHFSQNGVYGYNALNNMGKLYARHVNKDTPYQMSSTIRVYFKPKHIRVWVPRPPRLCPYIKSSNVNFEPTNLTDSRSSITYVPDTIRPEVRTAGKFGQQSGAIYVGNYRVVNRHLATHSDWQNCVWEEYNRDLLVSTTTAHGCDTIARCQCTTGVYFCASRNKHYPVTFEGPGLVEVQESEYYPKRYQS

SEQ ID NO: 83 - DNA, 660 bp, Coxsackievirus B
  CDS (1)..(660)
attctcacccaccaaataatgtatgtgccaccaggtggcccagtaccagataaggttgactcatatgtgtggcagacatccactaatcccagtgttttctggaccgaaggcaatgcgccgccacgcatgtccattccatttttgagcattggcaatgcatattcgaacttttatgatggctggtcggagtttgccagaagcggggtgtatggcataaacactttgaacaacatggggaccctttacgcaagacatgtgaatgctgggagcacaggaccaataaaaagcactattagaatctattttaaaccaaagcacgttaaggcgtggatacctagacctccaaggctgtgtcagtatgagaaagcaaagaatgttaattttcaacctagtggtgtaacaacaaccagacagagcatcacagcaatgacaaacactggggcgttcgggcaacaatcgggggcaatatacgtgggcaactatagagtggtgaatagacacctggccacattcacggactggcagaattgtgtgtgggaagactacaacagggatctacttgttagtactactacggcccatggatgtgatgtcatagctagatgccagtgcacattgggtgtatactactgtgcctcgaaaaataagcactatccagtc

SEQ ID NO: 84 - protein, 220 aa, Coxsackievirus B
ILTHQIMYVPPGGPVPDKVDSYVWQTSTNPSVFWTEGNAPPRMSIPFLSIGNAYSNFYDGWSEFARSGVYGINTLNNMGTLYARHVNAGSTGPIKSTIRIYFKPKHVKAWIPRPPRLCQYEKAKNVNFQPSGVTTTRQSITAMTNTGAFGQQSGAIYVGNYRVVNRHLATFTDWQNCVWEDYNRDLLVSTTTAHGCDVIARCQCTLGVYYCASKNKHYPV

SEQ ID NO: 85 - DNA, 744 bp, Echovirus
  CDS (1)..(744)
tggacagaaggcaatgcccctcccaggatgtcgataccattcatgagtgttggtaacgcatattgcaacttttatgatggatggtctcatttcagccagagcggtgtgtacggatacaccactttgaacaacatgggacacctgtattttagacatgtgaacaaatcaactgcatatccagttaacagtgttgcccgcgtctattttaagcccaagcacgtgaaggcatgggtgcctcgtgcgccacgcttgtgcccatatctgtatgcaagaaatgtcaactttgatgtgcaaggggtaaccgaatctcgaggcaaaattacccttgaccgatcaactcacaatcctttgtcaaacactggtgtttttgggcaacaatcaggggctgcatatgtgggcaactacagagttgtcaacagacacctcgccactcatgctgaccggcaaaattgcgtgtgggaggactacaatagagacttgttggttagcaccaccacagcccacgggtgtgacaccatagccaggtgccagtgctccacaggtgtgtatttttgcgcatccaggaacaaacattacccagtgtcatttgaagggcccgggctcgtcgaggtgcaggagagtgagtattacccaagaaggtaccaatcacatgtgttgctcgccgcagggttttctgagccaggtgactgtgggggcatcttgagatgcgagcatggcgtcatagggcttgtgaccatggggggc

SEQ ID NO: 86 - protein, 248 aa, Echovirus
WTEGNAPPRMSIPFMSVGNAYCNFYDGWSHFSQSGVYGYTTLNNMGHLYFRHVNKSTAYPVNSVARVYFKPKHVKAWVPRAPRLCPYLYARNVNFDVQGVTESRGKITLDRSTHNPLSNTGVFGQQSGAAYVGNYRVVNRHLATHADRQNCVWEDYNRDLLVSTTTAHGCDTIARCQCSTGVYFCASRNKHYPVSFEGPGLVEVQESEYYPRRYQSHVLLAAGFSEPGDCGGILRCEHGVIGLVTMGG

SEQ ID NO: 87 - DNA, 744 bp, Echovirus
  CDS (1)..(744)
tggacagaaggcaatgcccctcccaggatgtcgataccattcatgagtgttggtaacgcatattgcaacttttatgatggatggtctcatttcagccagagcggtgtgtacggatacactactttgaacaacatgggacacctgtattttagacatgtgaacaaatcaactgcatatccagttaacagtgttgcccgcgtctattttaagcccaagcacgtgaaggcatgggtgcctcgtgcgccacgcttgtgcccatatctgtatgcaagaaatgtcaactttgatgtgcaaggggtaaccgaatctcggggcaaaattacccttgaccgatcaactcacaatcctttgtcaaacactggtgtttttgggcaacaatcaggggctgcatatgtgggcaactacagagttgtcaacagacacctcgccactcatgctgaccggcagaattgcgtgtgggaggactacaatagagacttgttggttagcaccaccacagcccacgggtgtgacaccatagccaggtgccagtgctccacaggtgtgtatttttgcgcatccaggaacaaacattacccagtgtcatttgaagggcccgggctcgtcgaggtgcaggagagtgagtattacccaagaaggtaccaatcacatgtgttgctcgccgcagggttttctgagccaggtgactgtgggggcatcttgagatgcgagcatggcgtcatagggcttgtgaccatggggggc

SEQ ID NO: 88 - protein, 248 aa, Echovirus; identical to SEQ ID NO: 86

SEQ ID NO: 89 - DNA, 744 bp, Echovirus
  CDS (1)..(744)
tggacagaaggcaatgcccctcccaggatgtcgataccattcatgagtgttggtaacgcatattgcaacttttatgatggatggtctcatttcagccagagcggtgtgtacggatacactactttgaacaacatgggacacctgtattttagacatgtgaacaaatcaactgcatatccagttaacagtgttgcccgcgtctattttaagcccaagcacgtgaaggcatgggtgcctcgtgcgccacgcttgtgcccatatctgtatgcaagaaatgtcaactttgatgtgcaaggggtaaccgaatctcggggcaaaattacccttgaccgatcaactcacaatcctttgtcaaacactggtgtttttgggcaacaatcaggggctgcatatgtgggcaactacagagttgtcaacagacacctcgccactcatgctgactggcagaattgcgtgtgggaggactacaatagagacttgttggttagcaccaccacagcccacgggtgtgacaccatagccaggtgccagtgctccacaggtgtgtatttttgcgcatccaggaacaaacattacccagtgtcatttgaagggcccgggctcgtcgaggtgcaggagagtgagtattacccaagaaggtaccaatcacatgtgttgctcgccgcagggttttctgagccaggtgactgtgggggcatcttgagatgcgagcatggcgtcatagggcttgtgaccatggggggc

SEQ ID NO: 90 - protein, 248 aa, Echovirus
WTEGNAPPRMSIPFMSVGNAYCNFYDGWSHFSQSGVYGYTTLNNMGHLYFRHVNKSTAYPVNSVARVYFKPKHVKAWVPRAPRLCPYLYARNVNFDVQGVTESRGKITLDRSTHNPLSNTGVFGQQSGAAYVGNYRVVNRHLATHADWQNCVWEDYNRDLLVSTTTAHGCDTIARCQCSTGVYFCASRNKHYPVSFEGPGLVEVQESEYYPRRYQSHVLLAAGFSEPGDCGGILRCEHGVIGLVTMGG

SEQ ID NO: 91 - DNA, 747 bp, Echovirus
  CDS (1)..(747)
atcttttggacagaggggaacgcaccacctaggatgtctattcctttcattagcattggcaacgcgtatagcaacttctatgatggttggtcacacttctcccaaaacggtgtgtacgggtataacacacttaatcacatgggccagatttacgtcaggcacgtgaacgggtcttccccactgcctatgactagcacagtcaggatgtacttcaaaccaaagcatgtaaaggcatgggtcccacgcccgcctagattgtgtcagtacaagaacgcagcaaccgtcaactttacaccaaccaacatcaccgaaaagaggaatgacatcaattacataccagagacagtcagaccggacgtcgcaacgcatggggcgtttggccagcagtcaggtgctgcatatgttggcaactataggatagtaaacaggcatttagctacccgcactgattggaggaactgtgtgtgggaagactacaatagggaccttctagtgagcactactacggcacacggctgtgacaccattgccagatgccaatgcacaacgggtgtctatttctgtgcatcgaagaacaaacactatccagtacattttgaaggaccgggactggtagaagttcaagagagtgagtattacccaaagagataccaatcacacgtgctgttagctgcggggttttctgaaccgggagactgtggagggattcttagatgcgaacacggagttatcggccttgtcaccatg

SEQ ID NO: 92 - protein, 249 aa, Echovirus
IFWTEGNAPPRMSIPFISIGNAYSNFYDGWSHFSQNGVYGYNTLNHMGQIYVRHVNGSSPLPMTSTVRMYFKPKHVKAWVPRPPRLCQYKNAATVNFTPTNITEKRNDINYIPETVRPDVATHGAFGQQSGAAYVGNYRIVNRHLATRTDWRNCVWEDYNRDLLVSTTTAHGCDTIARCQCTTGVYFCASKNKHYPVHFEGPGLVEVQESEYYPKRYQSHVLLAAGFSEPGDCGGILRCEHGVIGLVTM

SEQ ID NO: 93 - DNA, 645 bp, Echovirus
  CDS (1)..(645)
gtgtcctggaccgagggtaatgccccgcctaggatatcaataccatttataagtgtgggaaatgcctacagctgtttttacgacggctggtcacacttctcgcagactggagtttatggttacaacacacttaatgatatgggccaactgtttgtaaggcatgtgaacgaggcaagtccgggtgctgtgtctagtgtggtgagaatatatttcaagcccaaacacatcaaggcatgggtacctagaccaccaaggttgtgccagtatgtcaatgcctctacagtaaacttcacgccagagggagtcaccaaatctcgaacggacttgatgactacaggggcctttggtcaacaatctggtgcagtgtatgtaggaaattacagagtggtaaatagacacctggctacacacgcagactggcaaaactgcgtgtgggaggactacaacagggacctccttgtgagcaccaccacagcccatggatgtgacgtcttagccaggtgccagtgcacgacaggggtgtacttctgccagtcgaggaacaagcattacctaattagtttcgagggtccgggacttgtcgaggtgcaggagagtgaatactatcccaaaaggtaccaatctcacgtgttacttgctgcaggc

SEQ ID NO: 94 - protein, 215 aa, Echovirus
VSWTEGNAPPRISIPFISVGNAYSCFYDGWSHFSQTGVYGYNTLNDMGQLFVRHVNEASPGAVSSVVRIYFKPKHIKAWVPRPPRLCQYVNASTVNFTPEGVTKSRTDLMTTGAFGQQSGAVYVGNYRVVNRHLATHADWQNCVWEDYNRDLLVSTTTAHGCDVLARCQCTTGVYFCQSRNKHYLISFEGPGLVEVQESEYYPKRYQSHVLLAAG

SEQ ID NO: 95 - DNA, 711 bp, Echovirus
  CDS (1)..(711)
atcttctggactgaaggtaacgcccccccacgcatgtccataccattcataagcatagggaacgcatacagtaatttttatgatgggtggtctcatttctcacagaatggagtgtacgggtacaatgcactaaacaacatgggcaagttatacgcacgtcatgtgaacaaagacacaccgtaccaaatgtccagcacaattcgcgtgtacttcaaacccaaacacgtaagggtttgggtaccgcgcccaccacgcctgtgtccatatattaagtccagtaatgtcaactttgaccctactaatttgaccgaccaaagacagagcataacgtatgtgcccgatactatccgcccagaggttcgcacagctggtaagtttggtcagcaatccggcgcaatttacgtagggaactacagggtagtaaatagacatctcgccacacataatgactggcaaaattgtgtgtgggaggactatggcagagatctccttgtgagcactactacggcgcacggatgtgacaccatagccaggtgccagtgtaccacaggagtgtacttctgtgcctcaaggaacaaacactacccgatttcatttgaagggccagggctggtggaagtccaggaaaatgagtactaccccaagagataccagtctcatgtactattggccgttggcttctccgagccgggtgattgtggcggcatcctc

SEQ ID NO: 96 - protein, 237 aa, Echovirus
IFWTEGNAPPRMSIPFISIGNAYSNFYDGWSHFSQNGVYGYNALNNMGKLYARHVNKDTPYQMSSTIRVYFKPKHVRVWVPRPPRLCPYIKSSNVNFDPTNLTDQRQSITYVPDTIRPEVRTAGKFGQQSGAIYVGNYRVVNRHLATHNDWQNCVWEDYGRDLLVSTTTAHGCDTIARCQCTTGVYFCASRNKHYPISFEGPGLVEVQENEYYPKRYQSHVLLAVGFSEPGDCGGIL